(19) United States
(12) Patent Application Publication
(10) Pub. No.: US 2009/0312336 A1
(43) Pub. Date: Dec. 17, 2009

(54) DIHYDROPTERIDINE COMPOUNDS AS ANTI PROLIFERATIVE AGENTS

(75) Inventors: Iain Simpson, Cheshire (GB); Richard Andrew Ward, Cheshire (GB)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/301,407

(22) PCT Filed: May 16, 2007
(86) PCT No.: PCT/GB2007/001806

§ 371 Date: Nov. 18, 2008

(30) Foreign Application Priority Data
May 19, 2006 (US) 60801754

(51) Int. Cl. A61K 31/519; C07D 487/04; A61P 35/00

(52) U.S. Cl. 514/249; 544/258

(57) ABSTRACT

Compounds of formula (I), or optionally the pharmacologically acceptable acid addition salts thereof, and their use in the inhibition of PLK activity are described.

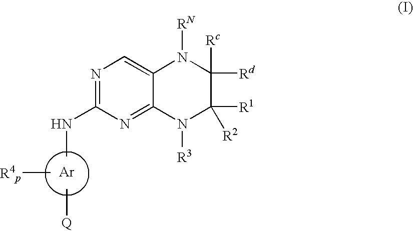

The present invention relates to pyrimidine derivatives, a process for their preparation, pharmaceutical compositions containing them, a process for preparing the pharmaceutical compositions, and their use in therapy and the treating of conditions mediated by polo-like kinases.

Many of the current treatment regimes for cell proliferation diseases such as cancer and psoriasis utilise compounds that inhibit DNA synthesis. Compounds that inhibit DNA synthesis may often prove to be toxic to many types of cells. However, the marked toxic effect on rapidly dividing cells such as tumour cells is often seen to offer a benefit in light of the general toxic nature of such compounds. Therefore, alternative antiproliferative agents that act by mechanisms other than the inhibition of DNA synthesis may offer the potential for selective targeting of the proliferating cells.

The Cyclin dependent kinase family (Cdks) have long been considered the master regulators of the cell cycle but an increasing number of diverse protein kinases are emerging as critical components of cell cycle progression. Among these are the polo-like kinase family (Plks), serine/threonine kinases that play multiple roles in regulating progress through cell cycle. In man, four distinct family members have been identified. These are Plk1, Plk2 (Snk), Plk3 (Fnk, Prk) and Plk4 (Sak).

The best characterized family member is Plk1 which is conserved from yeast to man and has been implicated in numerous mitotic processes including activation of Cdc25C and Cdk1/Cyclin B at the G2-M transition, centrosome maturation, spindle formation and assembly (Glover et al. 1998, Genes Dev. 12:3777-87; Barr et al 2004, Nat. Rev. Mol. Cell Biol 5:429-441). In the later stages of mitosis Plk1 is involved in separation of sister chromatids, activation of components of the anaphase-promoting complex and septin regulation during cytokinesis (van Vugt & Medema 2005, Oncogene 24:2844-2859).

Plk1 is overexpressed in a broad spectrum of cancer types including breast, colorectal, endometrial, oesophageal, ovarian, prostate, pancreatic, non small cell lung cancers and melanomas (Wolf et al. 1997, Oncogene 14:543-549; Knecht et al. 1999, Cancer Res. 59:2794-2797; Wolf et al. 2000, Pathol. Res. Pract. 196:753-759; Takahashi et al. 2003, Cancer Sci. 94:148-152). The expression of Plk1 often correlates with poor patient prognosis. The conclusion that Plk1 elevation is a cause and not a consequence of oncogenesis resulted from a study demonstrating that overexpression or constitutive expression of Plk1 induces malignant transformation of mammalian cells, causing tumour formation in nude mice (Smith et al 1997, Biochem. Biophys. Res. Commun 234:397-405)

Therapeutic potential for Plk1 inhibition has been demonstrated in studies employing both antisense oligonucleotides (ASO) and small molecule RNA (siRNA). Reduction in the level of Plk1 results in the inhibition of proliferation of tumour cells and loss of cell viability both in vivo and in vitro but does not inhibit proliferation of primary cells (Spankuch-Schmitt et al 2002, Oncogene 21: 3162-3171; Elez et al 2003, Oncogene 22:69-80). Microinjection of anti-Plk1 antibodies induced mitotic catastrophe in HeLa tumour cells. These cells displayed abnormal distribution of chromatin and monoastral microtubules while normal fibroblast cells arrested transiently in G2 phase of cell cycle as single mononucleated cells (Lane & Nigg 1996 J. Cell Biol. 135:1701-1713). These results suggest that Plk1 inhibition specifically targets cancer cells with checkpoint defects while cells with intact checkpoint pathways are less affected.

Although the exact functions of the other family members remains largely unknown, silencing of Plk2 in the presence of taxol or nocodazole significantly increases apoptosis suggesting Plk2 may prevent mitotic catastrophe following spindle damage (Bums et al. 2003, Mol Cell Biol 23: 5556-5571). Likewise silencing of Plk4 in mammalian cells induces apoptosis (Li et al. 2005, Neoplasia 7: 312-323) and plk4 null mouse embryos arrest with an increase in mitotic and apoptotic cells (Hudson et al. 2001, Curr Biol 11: 441-446).

Plk3 also appears to play roles in mitosis, like Plk1 it has been reported to phosphorylate Cdc25C, regulate microtubule dynamics and is involved in centrosome function. Overexpression of Plk3 has been observed in both breast and ovarian carcinomas, with little or no expression in adjacent normal tissue. Increased protein level was associated with enhanced mitosis and was significantly linked to reduced median survival time of patients (Weichert et al. 2005, Virchows Arch 446: 442-450; Weichert et al. 2004 Br. J. Cancer 90:815-821).

These findings suggest that pharmacological inhibitors of Plk family members should be of therapeutic value for treatment of proliferative disease including solid tumours such as carcinomas and sarcomas and the leukaemias and lymphoid malignancies. In addition Plk inhibitors should be useful in the treatment of other disorders associated with uncontrolled cellular proliferation.

Pteridinone derivatives are known from the prior art as active substances with an antiproliferative activity. WO 01/019825 and WO 03/020722 describe the use of pteridinone derivatives for the treatment of tumoural diseases.

The resistance of many types of tumours calls for the development of new pharmaceutical compositions for combating tumours.

The aim of the present invention is to provide new compounds having an antiproliferative activity.

According to a first aspect of the present invention there is provided a compound of formula (1):

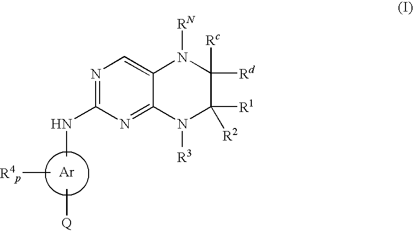

wherein
R¹, R² each independently represents hydrogen, an optionally substituted C₁₋₆alkyl group or an optionally substituted C₃₋₆cycloalkyl group, or R¹ and R² together with the carbon atom to which they are attached form a 3- to 6-membered saturated or unsaturated ring optionally comprising 1 to 2 heteroatoms;
R³ represents hydrogen, an optionally substituted C₁₋₁₂alkyl group, an optionally substituted C₂₋₁₂alkenyl group, an optionally substituted C₂₋₁₂alkynyl group, an optionally substituted C₆₋₁₄aryl group, an optionally substituted C₃₋₁₂cycloalkyl group, an optionally substituted C₃₋₁₂cycloalkenyl group, an optionally substituted C₇₋₁₂polycycloalkyl group, an optionally substituted C₇₋₁₂polycycloalkenyl group, an optionally substituted C₅₋₁₂spirocycloalkyl group, an optionally substituted C₃₋₁₂heterocycloalkyl group comprising 1 or 2 heteroatoms, or an optionally substituted C₃₋₁₂heterocycloalkenyl group comprising 1 or 2 heteroatoms;
R^{c}, R^{d} each independently represents hydrogen, an optionally substituted C₁₋₆alkyl group or an optionally substituted C₃₋₆cycloalkyl group, or R^{c} and R^{d} together with the carbon atom to which they are attached form a 3- to 6-membered saturated or unsaturated ring optionally comprising 1 to 2 heteroatoms; or
optionally one of R¹ and R³, or R² and R³, or R¹ and R^{c}, or R² and R^{d} together represent a saturated or unsaturated C₁₋₄alkyl bridge optionally comprising 1 heteroatom;
R⁴ each independently represent —CN, hydroxy, —NR⁶R⁷, halogen, an optionally substituted C₁₋₆alkyl group, an optionally substituted C₃₋₆cycloalkyl group, an optionally substituted C₂₋₆alkenyl group, an optionally substituted C₂₋₆alkynyl group, an optionally substituted C₁₋₅alkyloxy group, an optionally substituted C₃₋₆cycloalkyloxy group, an optionally substituted C₂₋₅alkenyloxy group, an optionally substituted C₂₋₅alkynyloxy group, an optionally substituted C₁₋₆alkythio group, an optionally substituted C₁₋₆alkylsulphoxo group or an optionally substituted C₁₋₆alkylsulphonyl group;
p is 0, 1 or 2;
Q¹ is —C(═X)—NR^{a}R^{b}, —NR^{a2}R^{b2}, —S(O)₂—NR^{a3}R^{b3}, S(O)ₖ—R^{a4}, —C(═X)—OR^{a5}, —OR^{a6};
k is 0, 1 or 2;
R^{a} represents H or an optionally substituted C₁₋₆alkyl group, and R^{b} represents -Lₙ-R⁵ₘ, or R^{a} and R^{b} together with the nitrogen atom to which they are attached form a 3- to 7-membered saturated or unsaturated heterocyclic ring optionally comprising 1 to 2 additional heteroatoms;
R^{a2} represents H or an optionally substituted C₁₋₆alkyl group, and R^{b2} represents -Lₙ-R⁵ₘ, or R^{a2} and R^{b2} together with the nitrogen atom to which they are attached form a 3- to 7-membered saturated or unsaturated heterocyclic ring optionally comprising 1 to 2 additional heteroatoms;
R^{a3} represents H or an optionally substituted C₁₋₆alkyl group, and R^{b3} represents -Lₙ-R⁵ₘ, or R^{a3} and R^{b3} together with the nitrogen atom to which they are attached form a 3- to 7-membered saturated or unsaturated heterocyclic ring optionally comprising 1 to 2 additional heteroatoms;
R^{a4} represents -Lₙ-R⁵ₘ;
R^{a5} represents -Lₙ-R⁵ₘ;
R^{a6} represents -Lₙ-R⁵ₘ;
L represents a linker selected from optionally substituted C₂₋₁₀alkyl, optionally substituted C₂₋₁₀alkenyl, optionally substituted C₆₋₁₄aryl, optionally substituted —C₂₋₄alkyl-C₆₋₁₄aryl, optionally substituted —C₆₋₁₄aryl-C₁₋₄alkyl, optionally substituted C₃₋₁₂cycloalkyl and optionally substituted heteroaryl comprising 1 or 2 nitrogen atoms;
n is 0 or 1
m is 1 or 2
R⁵ represents a group selected from among optionally substituted morpholinyl, piperidinyl, piperazinyl, piperazinylcarbonyl, pyrrolidinyl, tropenyl, diketomethylpiperazinyl, sulphoxomorpholinyl, sulphonylmorpholinyl, thiomorpholinyl, azacycloheptyl and —NR⁸R⁹;
R⁶, R⁷ each independently represents hydrogen or an optionally substituted C₁₋₄alkyl group;
R⁸, R⁹ each independently represents hydrogen, C₁₋₆alkyl, —C₁₋₄alkyl-C₃₋₁₀cycloalkyl, C₃₋₁₀cycloalkyl, C₆₋₁₄aryl, —C₁₋₄alkyl-C₆₋₁₄aryl, pyranyl, pyridinyl, pyrimidinyl, C₁₋₄alkyloxycarbonyl, C₆₋₁₄arylcarbonyl, C₁₋₄alkylcarbonyl, C₆₋₁₄arylmethyloxycarbonyl, C₆₋₁₄arylsulphonyl, C₁₋₄alkylsulphonyl or C₆₋₁₄aryl-C₁₋₄alkylsulphonyl;
X is O, S or H₂;
Ar represents a 5- or 6-membered aromatic or heteroaromatic ring optionally comprising at least one ring heteroatom selected from nitrogen, oxygen and sulphur; and
R^{N} represents hydrogen, —NH₂, —OH, —CN, —C≡CH, —C(═O)NH₂, C₁₋₃alkyl, C-₁₋₃alkylamino, C₁₋₃alkylthio or C₁₋₃alkyloxy,
   or optionally the pharmacologically acceptable acid addition salts thereof.

The term alkyl group, including alkyl groups which are a part of other groups, unless otherwise stated, includes branched and unbranched alkyl groups with 1 to 12 carbon atoms. Examples of C₁₋₁₂alkyl groups include methyl, ethyl, propyl, butyl, pentyl, hexyl, heptyl, octyl, nonyl, decyl and dodecyl groups. Unless otherwise stated, the terms propyl, butyl, pentyl, hexyl, heptyl, octyl, nonyl, decyl and dodecyl include all the possible isomeric forms. For example, the term propyl includes the two isomeric groups n-propyl and iso-propyl, the term butyl includes n-butyl, iso-butyl, sec-butyl and tert-butyl, the term pentyl includes iso-pentyl, neopentyl, etc.

In the above mentioned alkyl groups, one or more hydrogen atoms may optionally be replaced by other substituent groups. For example, alkyl groups may be substituted by the following substituents groups: ═O; OH; NO₂; CN; —NH₂; halogen, for example fluorine or chlorine; optionally substituted C₁₋₁₀alkyl, for example methyl, ethyl, propyl, trifluoromethyl; optionally substituted —OC₁₋₃alkyl, for example OMe, OEt, —OCHF₂, —OCF₃; —COOH; —COO—C₁-C₄alkyl, for example —COOMe or —COOEt; or —CONH₂. “═O” denotes an oxygen atom linked via a double bond. All the hydrogen atoms of the alkyl group may optionally be replaced by substituent groups, for example a trifluoromethyl group is a methyl group wherein all the hydrogen atoms have been replaced with fluorine atoms.

The term alkyl bridge, unless otherwise stated, includes branched and unbranched alkyl bridging groups with 1 to 5 carbon atoms, for example methylene, ethylene, propylene, butylene and pentylene bridges. Unless otherwise stated, the terms propylene, butylene and pentylene include all the possible isomeric forms. In the aforementioned alkyl bridges, 1 or 2 C-atoms may optionally be replaced by one or more heteroatoms selected from among oxygen, nitrogen or sulphur.

The term alkenyl groups (including those which are a part of other groups), unless otherwise stated, includes branched and unbranched alkylene groups with 2 to 10 carbon atoms comprising at least one carbon-carbon double bond. Examples of C₂₋₁₀alkenyl groups include ethenyl, propenyl, butenyl, pentenyl, hexenyl, heptenyl, octenyl nonenyl and decenyl groups. Unless otherwise stated, the abovementioned terms propenyl, butenyl, pentenyl, hexenyl, heptenyl, octenyl, nonenyl and decenyl also include all the possible isomeric forms. For example, the term butenyl includes 1-butenyl, 2-butenyl, 3-butenyl, 1-methyl-1-propenyl, 1-methyl-2-propenyl, 2-methyl-1-propenyl, 2-methyl-2-propenyl and 1-ethyl-1-ethenyl.

In the above mentioned alkenyl groups, unless otherwise stated, one or more hydrogen atoms may optionally be replaced by other substituent groups. For example, alkenyl groups may be substituted by the following substituents groups: ═O; OH; NO₂; CN; —NH₂; halogen, for example fluorine or chlorine; optionally substituted C₁₋₁₀alkyl, for example methyl, ethyl, propyl, trifluoromethyl; optionally substituted —OC₁₋₃alkyl, for example OMe, OEt, —OCHF₂, —OCF₃; —COOH; —COO—C₁-C₄alkyl, for example —COOMe or —COOEt; or —CONH₂. “═O” denotes an oxygen atom linked via a double bond. All the hydrogen atoms of the alkenyl group may optionally be replaced, for example a trifluoroethylene group is an ethylene group wherein all the hydrogen atoms have been replaced with fluorine atoms.

The term alkynyl groups (including those which are a part of other groups), unless otherwise stated, includes branched and unbranched alkynyl groups with 2 to 10 carbon atoms comprising at least one triple bond. Examples of C₂₋₁₀alkynyl groups include ethynyl, propynyl, butynyl, pentynyl, hexynyl, heptynyl, octynyl, nonynyl and decynyl groups. Unless otherwise stated, the terms propynyl, butynyl, pentynyl, hexynyl, heptynyl, octynyl, nonynyl and decynyl also include all the possible isomeric forms. For example, the term butynyl includes 1-butynyl, 2-butynyl, 3-butynyl and 1-methyl-2-propynyl.

In the above mentioned alkynyl groups, unless otherwise stated, one or more hydrogen atoms may optionally be replaced by other substituent groups. For example, alkynyl groups may be substituted by the following substituents groups: ═O; OH; NO₂; CN; —NH₂; halogen, for example fluorine or chlorine; optionally substituted C₁₋₁₀alkyl, for example methyl, ethyl, propyl, trifluoromethyl; optionally substituted —OC₁₋₃alkyl, for example OMe, OEt, —OCHF₂, —OCF₃; —COOH; —COO—C₁-C₄alkyl, for example —COOMe or —COOEt; or —CONH₂. “═O” denotes an oxygen atom linked via a double bond. All the hydrogen atoms of the alkynyl group may optionally also be replaced.

The term aryl includes aromatic ring systems with 6 to 14 carbon atoms, said aromatic ring systems comprising one or more rings having from 6 to 14 ring atoms wherein at least one ring is aromatic. Examples of C₆₋₁₄aryl groups include phenyl (C₆), indenyl (C₉), naphthyl (C₁₀), fluorenyl (C₁₃), anthracyl (C₁₄), and phenanthryl (C₁₄). In the above mentioned aryl groups, unless otherwise stated, one or more hydrogen atoms may optionally be replaced by other substitutent groups. For example, aryl groups may be substituted by the following substituents groups: OH; NO₂; CN; NH₂; halogen, for example fluorine or chlorine; optionally substituted C₁₋₁₀alkyl, for example methyl, ethyl, propyl or CF₃; optionally substituted —OC₁₋₃alkyl, for example —OMe, —OEt, OCHF₂, or OCF₃; —COOH, —COO—C₁-C₄alkyl, for example —COOMe or —COOEt, or —CONH₂.

The term heteroaryl comprising 1 or 2 nitrogen atoms includes heteroaromatic ring systems with 5 to 14 ring atoms, said heteroaromatic ring systems comprising one or more rings having from 5 to 14 ring atoms wherein at least one ring is aromatic and wherein one or two of the ring atoms are replaced by nitrogen atoms the remaining ring atoms being carbon atoms. Examples of heteroaryl groups wherein up to two carbon atoms are replaced by one or two nitrogen atoms comprising one ring include pyrrolyl, pyrazolyl, imidazolyl, triazolyl, pyridinyl and pyrimidinyl groups. Each of the aforementioned examples of heteroaryl rings may optionally also be anellated by a further ring, for example a benzene ring. Examples of heteroaryl groups wherein up to two carbon atoms are replaced by one or two nitrogen atoms comprising two rings include indolyl, benzimidazolyl, quinolinyl, isoquinolinyl and quinazolinyl. In the above mentioned heteroaryl groups, unless otherwise stated, one or more hydrogen atoms may optionally be replaced by other substituent groups. For example, heteroaryl groups may be substituted by the following substituents groups: F; Cl; Br; OH; OMe; Me; Et; CN; NH₂; CONH₂; optionally substituted phenyl; and optionally substituted heteroaryl, for example optionally substituted pyridyl.

The term cycloalkyl groups, unless otherwise stated, includes cycloalkyl groups comprising 1 ring with 3-12 carbon atoms. Examples of C₃₋₁₂cycoalkyl groups include cyclopropyl, cyclobutyl, cyclopentyl, cyclohexyl, cycloheptyl, cyclooctyl, cyclononyl, cyclodecyl, cycloundecyl and cyclododecyl groups. In the abovementioned cycloalkyl groups, unless otherwise stated, one or more hydrogen atoms may optionally be replaced by other substituent groups. For example, cycloalkyl groups may be substituted by the following substituents groups: ═O; OH; NO₂; CN; —NH₂; halogen, for example fluorine or chlorine; optionally substituted C₁₋₁₀alkyl, for example methyl, ethyl, propyl, trifluoromethyl; optionally substituted —OC₁₋₃alkyl, for example OMe, OEt, —OCHF₂, —OCF₃; —COOH; —COO—C₁-C₄alkyl, for example —COOMe or —COOEt; or —CONH₂. “═O” denotes an oxygen atom linked via a double bond.

The term cycloalkenyl, unless otherwise stated, includes cycloalkenyl groups with 3-12 carbon atoms comprising one ring, said ring comprising at least one carbon-carbon double bond. Examples of C₃₋₁₂cycloakenyl groups include cyclopropenyl, cyclobutenyl, cyclopentenyl, cyclohexenyl, cycloheptenyl, cyclooctenyl, cyclononenyl, cyclodecenyl, cycloundecenyl and cyclododecenyl groups. In the abovementioned cycloalkenyl groups, unless otherwise stated, one or more hydrogen atoms may optionally be replaced by other substituent groups. For example, cycloalkenyl groups may be substituted by the following substituent groups: ═O; OH; NO₂; CN; —NH₂; halogen, for example fluorine or chlorine; optionally substituted C₁₋₁₀alkyl, for example methyl, ethyl, propyl, trifluoromethyl; optionally substituted —OC₁₋₃alkyl, for example OMe, OEt, —OCHF₂, —OCF₃; —COOH; —COO—C₁-C₄alkyl, for example —COOMe or —COOEt; or —CONH₂. “═O” denotes an oxygen atom linked via a double bond.

The terms heterocycloalkyl and heterocycloakenyl, unless otherwise described in the definitions, includes 3- to 12-membered, for example 5-, 6- or 7-membered, heterocycles which may contain 1 to 4 heteroatoms selected from nitrogen, oxygen or sulphur. Heterocycloalkyl denotes a saturated heterocycle, and heterocycloakenyl denotes an unsaturated heterocycle. Examples of heterocycloalkyl or heterocycloakenyl groups include tetrahydrofuran, tetrahydrofuranone, gamma-butyrolactone, alpha-pyran, gamma-pyran, dioxolane, tetrahydropyran, dioxane, dihydrothiophene, thiolan, dithiolan, pyrroline, pyrrolidine, pyrazoline, pyrazolidine, imidazoline, imidazolidine, tetrazole, piperidine, pyridazine, pyrimidine, pyrazine, piperazine, triazine, tetrazine, morpholine, thiomorpholine, diazepan, oxazine, tetrahydro-oxazinyl, isothiazole, and pyrazolidine. In the abovementioned heterocycloalkyl or heterocycloakenyl groups, unless otherwise stated, one or more hydrogen atoms may optionally be replaced by other substituent groups. For example, heterocycloalkyl or heterocycloakenyl groups may be substituted by the following substituents groups: ═O; OH; CN; —NH₂; halogen, for example fluorine or chlorine; optionally substituted C₁₋₄alkyl, for example methyl, ethyl, propyl, trifluoromethyl; optionally substituted —OC₁₋₃alkyl, for example OMe, OEt, —OCHF₂, —OCF₃; —COOH; —COO—C₁-C₄alkyl, for example —COOMe or —COOEt; or —CONH₂. “═O” denotes an oxygen atom linked via a double bond.

The term polycycloalkyl, unless otherwise stated, includes cycloalkyl groups comprising 3 to 12 carbon atoms and comprising 2 or more rings. Examples of polycycloalkyl groups include optionally substituted, bi-, tri-, tetra- or pentacyclic cycloalkyl groups, for example pinane, 2,2,2-octane, 2,2,1-heptane or adamantane.

The term polycycloalkenyl, unless otherwise stated, includes cycloalkenyl groups comprising 7 to 12 carbon atoms and comprising 2 or more rings wherein at least one ring comprises a carbon-carbon double bond. Examples of polycycloalkenyl groups are optionally bridged and/or substituted bi-, tri-, tetra- or pentacyclic cycloalkenyl groups, for example bicycloalkenyl or tricycloalkenyl groups having at least one double bond, such as norbornene.

The term spirocycloalkyl unless otherwise stated, includes spirocycloalkyl groups comprising 5 to 12 carbon atoms and comprising 2 or more rings wherein two rings are joined at a spiro carbon centre. Examples of spirocycloalkyl groups include spiro[4.4]nonyl and spiro[3.4]octyl.

The term 5- or 6-membered aromatic or heteroaromatic ring optionally comprising at least one ring heteroatom selected from nitrogen, oxygen and sulphur is a fully unsaturated, aromatic monocyclic ring containing 5 or 6 atoms of which one or more ring atoms is optionally a heteroatom selected from nitrogen, oxygen or sulphur, with the remaining ring atoms being carbon. Examples of a 5- or 6-membered aromatic or heteroaromatic ring optionally comprising at least one ring heteroatom selected from nitrogen, oxygen and sulphur include furyl, imidazolyl, isothiazolyl, isoxazolyl, oxaxolyl, phenyl, pyrazinyl, pyrazolyl, pyridazinyl, pyridyl, pyrimidinyl, pyrrolyl thiazolyl, thienyl and triazolyl rings.

The term 3- to 6-membered saturated or unsaturated ring optionally comprising 1 to 2 heteroatoms includes optionally substituted C₃₋₆cylcoalkyl and optionally substituted C₃₋₆cylcoalkenyl groups, and optionally substituted C₃₋₆heterocylcoalkyl and optionally substituted C₃₋₆heterocylcoalkenyl groups each with 1 or 2 heteroatoms.

The term halogen includes fluorine, chlorine, bromine or iodine.

The terms alkyloxy (—OR wherein R is an alkyl), alkenyloxy (—OR wherein R is an alkenyl), alkynyloxy (—OR wherein R is an alkynyl) and cycloalkyloxy (—OR wherein R is a cycloalkyl) denote an —OR group wherein the respective alkyl, alkenyl, alkynyl or cycloalkyl group is as hereinbefore described above.

The terms alkylthio, alkylsulphoxo and alkylsulphono denotes an —S(O)ₓR group wherein x=0, 1 or 2 respectively and R is an alkyl group as hereinbefore described above.

The term -alkyl-aryl refers to an alkyl group with an aryl substituent. The term -alkyl-cycloalkyl refers to an alkyl group with a cycloakyl substituent. The term -aryl-alkyl refers to an aryl group with an alkyl substituent. The terms alkoxycarbonyl (—(C═)OR), alkylcarbonyl (—COR) and arylcarbonyl (—COR) refer to a carbonyl group with an alkoxy, alkyl or aryl substituent.

When R⁵ represents a substituted morpholinyl, piperidinyl, piperazinyl, piperazinylcarbonyl, pyrrolidinyl, tropenyl, diketomethylpiperazinyl, sulphoxomorpholinyl, sulphonylmorpholinyl, thiomorpholinyl, or azacycloheptyl, one or more substituents may be present and are as defined above for R⁸.

All the groups mentioned in the definition of R¹ to R⁹ may optionally be branched and/or substituted.

According to a second aspect of the present invention there is provided a compound of formula (II):

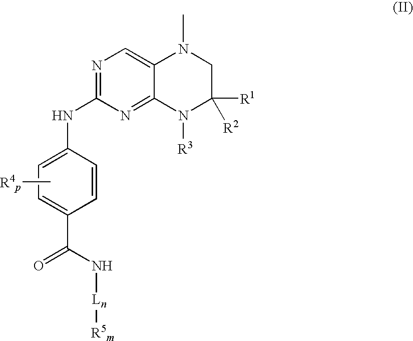

wherein
R¹, R² each independently represents hydrogen or an optionally substituted C₁₋₆alkyl group, or
R¹ and R² together with the carbon atom to which they are attached form a 3- to 6-membered saturated or unsaturated ring optionally comprising 1 to 2 heteroatoms;
R³ represents hydrogen, an optionally substituted C₁₋₁₂alkyl group, an optionally substituted C₂₋₁₂alkenyl group, an optionally substituted C₂₋₁₂alkynyl group, an optionally substituted C₆₋₁₄aryl group, an optionally substituted C₃₋₁₂cycloalkyl group, an optionally substituted C₃₋₁₂cycloalkenyl group, an optionally substituted C₇₋₁₂polycycloalkyl group, an optionally substituted C₇₋₁₂polycycloalkenyl group, an optionally substituted C₅₋₁₂spirocycloalkyl group, an optionally substituted C₃₋₁₂heterocycloalkyl group comprising 1 or 2 heteroatoms, or an optionally substituted C₃₋₁₂heterocycloalkenyl group comprising 1 or 2 heteroatoms, or
R¹ and R³ or R² and R³ together represent a saturated or unsaturated C₃₋₄alkyl bridge optionally comprising 1 heteroatom;
R⁴ each independently represent —CN, hydroxy, —NR⁶R⁷, halogen, an optionally substituted C₁₋₆alkyl group, an optionally substituted C₃₋₆cycloalkyl group, an optionally substituted C₂₋₆alkenyl group, an optionally substituted C₂₋₆alkynyl group, an optionally substituted C₁₋₅alkyloxy group, an optionally substituted C₃₋₆cycloalkyloxy group, an optionally substituted C₂₋₅alkenyloxy group, an optionally substituted C₂₋₅alkynyloxy group, an optionally substituted C₁₋₆alkythio group, an optionally substituted C₁₋₆alkylsulphoxo group or an optionally substituted C₁₋₆alkylsulphonyl group;
p is 0, 1 or 2;
L represents a linker selected from optionally substituted C₂₋₁₀alkyl, optionally substituted C₂₋₁₀alkenyl, optionally substituted C₆₋₁₄aryl, optionally substituted —C₂₋₄alkyl-C₆₋₁₄aryl, optionally substituted —C₆₋₁₄aryl-C₁₋₄alkyl, optionally substituted C₃₋₁₂cycloalkyl and optionally substituted heteroaryl comprising 1 or 2 nitrogen atoms;
n is 0 or 1
m is 1 or 2
R⁵ represents a group selected from among optionally substituted morpholinyl, piperidinyl, piperazinyl, piperazinylcarbonyl, pyrrolidinyl, tropenyl, diketomethylpiperazinyl, sulphoxomorpholinyl, sulphonylmorpholinyl, thiomorpholinyl, azacycloheptyl and —NR⁸R⁹;
R⁶, R⁷ each independently represents hydrogen or an optionally substituted C₁₋₄alkyl group; and
R⁸, R⁹ each independently represents hydrogen, C₁₋₆alkyl, —C₁₋₄alkyl-C₃₋₁₀cycloalkyl, C₃₋₁₀cycloalkyl, C₆₋₁₄aryl, —C₁₋₄alkyl-C₆₋₁₄aryl, pyranyl, pyridinyl, pyrimidinyl, C₁₋₄alkyloxycarbonyl, C₆₋₁₄arylcarbonyl, C₁₋₄alkylcarbonyl, C₆₋₁₄arylmethyloxycarbonyl, C₆₋₁₄arylsulphonyl, C₁₋₄alkylsulphonyl and C₆₋₁₄aryl-C₁₋₄alkylsulphonyl,
   or optionally the pharmacologically acceptable acid addition salts thereof.

In one embodiment, for compounds of the first and second aspects, the groups R¹ and R² may be identical or different and represent hydrogen or a C₁-C₆alkyl group optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino.

In another embodiment, for compounds of the first and second aspects, the groups R¹ and R² may be identical or different and represent hydrogen or a methyl or ethyl group.

In another embodiment, for compounds of the first and second aspects, the groups R¹ and R² are different wherein one of R¹ or R² represents hydrogen and the other represents a methyl or ethyl group.

In another embodiment, for compounds of the first and second aspects, R¹ and R² together represent a 2- to 5-membered alkyl bridge optionally comprising 1 to 2 heteroatoms selected from oxygen or nitrogen and optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino.

In another embodiment, for compounds of the first and second aspects, R¹ and R² together represent an ethylene or propylene bridge.

In another embodiment, for compounds of the first and second aspects, R³ represents hydrogen; a C₁-C₁₂alkyl, for example ethyl, propyl, butyl, pentyl or hexyl, optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; a C₂-C₁₂alkenyl, for example C₅-C₇alkenyl, optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; C₂-C₁₂alkynyl, for example C₅-C₇alkynyl, optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; a C₆-C₁₄aryl, for example phenyl, optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; a C₃-C₁₂cycloalkyl, for example cyclopentyl or cyclohexyl, optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; a C₃-C₁₂cycloalkenyl, for example C₅-C₇cycloalkenyl, optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; C₇-C₁₂polycycloalkyl optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; C₇-C₁₂polycycloalkenyl optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; C₅-C₁₂spirocycloalkyl optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; C₃-C₁₂heterocycloalkyl which contains 1 to 2 heteroatoms selected from oxygen, nitrogen or sulphur, for example pyranyl or piperinyl, pyrrolidinyl, pyrazinyl or morpholinyl, optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; and C₃-C₁₂heterocycloalkenyl which contains 1 to 2 heteroatoms selected from oxygen, nitrogen or sulphur, optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino.

In another embodiment, for compounds of the first and second aspects, R³ represents isopropyl, isobutyl, isopentyl, cyclopentyl, phenyl or cyclohexyl.

In another embodiment, for compounds of the first and second aspects, R¹ and R³ or R² and R³ together represent a saturated or unsaturated C₃-C₄alkyl bridge optionally comprising 1 heteroatom selected from oxygen or nitrogen.

In another embodiment, for compounds of the first and second aspects, when p is 1, R⁴ represents a group selected from among —CN; hydroxyl; —NR⁶R⁷; halogen, for example chlorine or fluorine; C₁-C₆alkyl, for example methyl, ethyl or propyl, optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; C₂-C₆alkenyl, for example ethenyl or propenyl, optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; C2-C₆alkynyl, for example ethynyl, propynyl or butynyl, optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; C₁-C₅alkyloxy, for example methoxy, ethoxy or propargyloxy, optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; C₂-C₅alkenyloxy optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; C₂-C₅alkynyloxy optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; C₁-C₆alkylthio optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; C₁-C₆alkylsulphoxo optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino and C₁-C₆alkylsulphonyl optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino.

In another embodiment, for compounds of the first and second aspects, when p is 1, R⁴ represents methoxy, methyl, ethoxy, ethyl, propargyloxy, chlorine.

In another embodiment, for compounds of the first and second aspects, when p is 2, each R⁴ may be the same or different and selected from methoxy, methyl, ethoxy, ethyl, propargyloxy, chlorine or fluorine.

In another embodiment, for compounds of the first and second aspects, when p is 2 and when each R⁴ is adjacent, both R⁴ together with the aromatic ring atoms to which they are attached form a 4- to 7-member unsaturated ring optionally comprising 1 to 2 heteroatoms.

In another embodiment, for compounds of the first and second aspects, L represents a linker selected from among C₂-C₁₀alkyl, for example ethyl, propyl, butyl or pentyl, optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; C₂-C₁₀alkenyl, optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; C₆-C₁₄aryl, for example phenyl, optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; —C₂-C₄alkyl-C₆-C₁₄aryl optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; —C₆-C₁₄aryl-C₁-C₄alkyl optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; C₃-C₁₂cycloalkyl, for example cyclohexyl, optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; and heteroaryl which contains 1 or 2 nitrogen atoms optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino.

In another embodiment, for compounds of the first and seconds aspects, when n is 1, L represents an optionally substituted a C₂₋₁₀alkyl linker.

In another embodiment, for compounds of the first and seconds aspects, when n is 1, L represents —C(CH₃)₂—CH₂— or —CH₂—C(CH₃)₂—CH₂—.

In another embodiment, for compounds of the first and second aspects, m is 1.

In another embodiment, for compounds of the first and second aspects, R⁵ represents a group selected from among optionally substituted morpholinyl, piperidinyl, piperazinyl, piperazinylcarbonyl, pyrrolidinyl, tropenyl, diketomethylpiperazinyl, sulphoxomorpholinyl, sulphonylmorpholinyl, thiomorpholinyl, —NR⁸R⁹ and azacycloheptyl wherein each morpholinyl, piperidinyl, piperazinyl, piperazinylcarbonyl, pyrrolidinyl, tropenyl, diketomethylpiperazinyl, sulphoxomorpholinyl, sulphonylmorpholinyl, thiomorpholinyl, —NR⁸R⁹ and azacycloheptyl is optionally substituted by one or more groups as defined for R⁸.

In another embodiment, for compounds of the first and second aspects, R⁵ represents piperidinyl, morpholinyl, pyrrolidinyl, sulphoxomorpholiny, piperazinyl, thiomorpholinyl or tropenyl each optionally substituted by one or more groups as defined for R⁸.

In another embodiment, for compounds of the first and second aspects, the groups R⁶ and R⁷ may be identical or different and represent hydrogen or C₁-C₄alkyl, for example methyl or ethyl.

In another embodiment, for compounds of the first and second aspects, the groups R⁸ and R⁹ may be identical or different and represent hydrogen; a C₁-C₆alkyl, for example methyl, ethyl or propyl, optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; —C₁-C₄alkyl-C₃-C₁₀cycloalkyl, for example —CH₂-cyclopropyl, optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; C₃-C₁₀cycloalkyl optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; C₆-C₁₄aryl, for example phenyl, optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; —C₁-C₄alkyl-C₆-C₁₄aryl, for example benzyl optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; pyranyl optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; pyridinyl optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; pyrimidinyl optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; pyranyl optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; C₁-C₄alkyloxycarbonyl optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; C₆-C₁₄arylcarbonyl optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; C₁-C₄alkylcarbonyl optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; C₆-C₁₄ arylmethyloxycarbonyl optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; C₆-C₁₄arylsulphonyl optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino; C₁-C₄alkylsulphonyl optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino and C₆-C₁₄aryl-C₁-C₄alkylsulphonyl optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino.

In another embodiment, for compounds of the first and second aspects, R⁸ represents methyl, ethyl or propyl.

In another embodiment, for compounds of the first and second aspects, R⁹ represents methyl, ethyl or propyl.

In another embodiment, for the compound of formula (I), R^{N} represents C₁₋₃alkyl.

In another embodiment, for the compound of formula (I), R^{N} represents methyl or ethyl.

In another embodiment, for the compound of formula (I), Q represents —C(═X)—NR^{a}R^{b} and X is O or CH₂.

In another embodiment, for the compound of formula (I) or (II), n=1 is 1 and L is an optionally substituted C₂₋₁₀alkyl linker.

In a further embodiment of the invention there is provided a subset of the compounds of formula (II) wherein R¹ to R⁴, R⁶ and R⁷ are as hereinbefore defined; and L represents a linker selected from among optionally substituted C₂₋₁₀alkyl, optionally substituted C₂₋₁₀alkenyl, optionally substituted C₆₋₁₄aryl, optionally substituted —C₂₋₄alkyl-C₆₋₁₄aryl, optionally substituted —C₆₋₁₄aryl-C₁₋₄alkyl, optionally substituted C₃₋₁₂cycloalkyl and optionally substituted heteroaryl comprising 1 or 2 nitrogen ring atoms; n denotes 1; m denotes 1 or 2; R⁵ denotes a group which is bound to L via a nitrogen atom, selected from optionally substituted morpholinyl, optionally substituted piperidinyl, optionally substituted piperazinyl, optionally substituted pyrrolidinyl, optionally substituted tropenyl, optionally substituted diketomethylpiperazinyl, optionally substituted sulphoxomorpholinyl, optionally substituted sulphonylmorpholinyl, optionally substituted thiomorpholinyl, —NR⁸R⁹ and optionally substituted azacycloheptyl; R⁸, R⁹ independently represent hydrogen, C₁₋₆alkyl, —C₁₋₄alkyl-C₃₋₁₀cycloalkyl, C₃₋₁₀cycloalkyl, C₆₋₁₄aryl, —C₁₋₄alkyl-C₆₋₁₄aryl, pyranyl, pyridinyl, pyrimidinyl, C₁₋₄alkyloxycarbonyl, C₆₋₁₄arylcarbonyl, C₁₋₄alkylcarbonyl, C₆₋₁₄arylmethyloxycarbonyl, C₆₋₁₄arylsulphonyl, C₁₋₄alkylsulphonyl and C₆₋₁₄aryl-C₁₋₄alkylsulphonyl, or optionally the pharmacologically acceptable acid addition salts thereof.

In a still further embodiment of the invention there is provided another subset of the compounds of formula (II) wherein R¹ to R⁴, R⁶ and R⁷ are as hereinbefore defined; L represents a linker selected from optionally substituted C₂₋₁₀alkyl, optionally substituted C₂₋₁₀alkenyl, optionally substituted C₆₋₁₄aryl, optionally substituted —C₂₋₄alkyl-C₆₋₁₄aryl, optionally substituted —C₆₋₁₄aryl-C₁₋₄alkyl, optionally substituted C₃₋₁₂cycloalkyl and optionally substituted heteroaryl comprising 1 or 2 nitrogen ring atoms; n denotes 0 or 1; m denotes 1 or 2; R⁵ denotes a group which is bound to L via a carbon atom, selected from among piperidinyl, piperazinyl, pyrrolidinyl, piperazinylcarbonyl, tropenyl, morpholinyl and azacycloheptyl each optionally substituted by one or more groups as defined for R⁸; and R⁸, R⁹ independently represent hydrogen, C₁₋₆alkyl, —C₁₋₄alkyl-C₃₋₁₀cycloalkyl, C₃₋₁₀cycloalkyl, C₆₋₁₄aryl, —C₁₋₄alkyl-C₆₋₁₄aryl, pyranyl, pyridinyl, pyrimidinyl, C₁₋₄alkyloxycarbonyl, C₆₋₁₄arylcarbonyl, C₁₋₄alkylcarbonyl, C₆₋₁₄arylmethyloxycarbonyl, C₆₋₁₄arylsulphonyl, C₁₋₄alkylsulphonyl and C₆₋₁₄aryl-C₁₋₄alkylsulphonyl, or optionally the pharmacologically acceptable acid addition salts thereof.

In an additional embodiment of the invention there is provided an additional subset of the compounds of formula (II) wherein L, m, n and R³ to R⁹ are as hereinbefore defined; and R¹, R² each independently represents hydrogen, Me, Et, Pr, or R¹ and R² together with the carbon atom to which they are attached form a 3- to 5-membered cycloalkyl ring, or optionally the pharmacologically acceptable acid addition salts thereof.

In a further additional embodiment of the invention there is provided an additional subset of the compounds of formula (II) wherein R¹, R², m, n and R⁵ to R⁸ are as hereinbefore defined; and R³ represents an optionally substituted C₁₋₁₀alkyl, optionally substituted C₃₋₇cycloalkyl, optionally substituted C₃₋₆heterocycloalkyl or optionally substituted C₆₋₁₄aryl group or R¹ and R³ or R² and R³ together represent a saturated or unsaturated C₃₋₄alkyl bridge optionally comprising 1 or 2 heteroatoms; R⁴ represents hydrogen, OMe, OH, Me, Et, Pr, OEt, NHMe, NH₂, F, CL, Br, O-propargyl, O-butynyl, CN, SMe, NMe₂, CONH₂, ethynyl, propynyl, butynyl and allyl; and L denotes a linker selected from among optionally substituted phenyl, phenylmethyl, cyclohexyl and branched C₁₋₆alkyl, or optionally the pharmacologically acceptable acid addition salts thereof.

In a further aspect of the invention, particular compounds of the invention are any one of Examples 1, 2, 3, 4, 5, 6, 7 and 8 or optionally the pharmacologically acceptable acid addition salts thereof.

It is understood that when referring to compounds of the first and second aspects of the invention or further embodiments thereof, such references are intended to include tautomers, the individual optical isomers, diastereomers or racemates and mixtures of the individual enantiomers, diastereomers or racemates of the compounds.

The compounds according to the first and second aspects of the invention may be present in the form of the individual optical isomers, mixtures of the individual enantiomers, diastereomers or racemates, in the form of the tautomers and also in the form of the free bases or the corresponding acid addition salts with pharmacologically acceptable acids, such as for example acid addition salts with hydrohalic acids, for example hydrochloric or hydrobromic acid, or organic acids, such as for example oxalic, fumaric, diglycolic or methanesulphonic acid.

The compounds of formula (I) or (II) above may be converted to a pharmaceutically acceptable salt, preferably an acid addition salt such as a hydrochloride, hydrobromide, phosphate, acetate, fumarate, maleate, tartrate, citrate, oxalate, methanesulphonate or p-toluenesulphonate, or an alkali metal salt such as a sodium or potassium salt.

Certain compounds of formula (I) or (II) are capable of existing in stereoisomeric forms. It will be understood that the invention encompasses the use of all geometric and optical isomers (including atropisomers) of the compounds of formula (I) or (II) and mixtures thereof including racemates. The use of tautomers and mixtures thereof also form an aspect of the present invention. The use of solvates of any of the compounds of formula (I) or (II) also forms an aspect of the present invention.

The invention also relates to a process for preparing a compound of general formula (II),

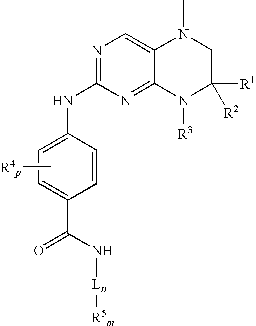

wherein R¹-R⁵, m, n and L are as hereinbefore defined, comprising reacting a compound of general formula (III)

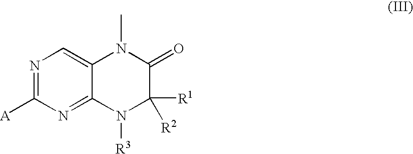

wherein R¹-R³ are as hereinbefore defined and A is a leaving group, with an optionally substituted compound of general formula (IV):

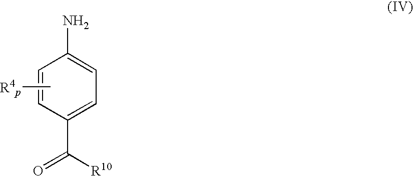

wherein R⁴ is as hereinbefore defined; and R¹⁰ denotes OH, NH-Lₘ-R⁵ₙ, OMe, OEt, to give a product of general formula (V)

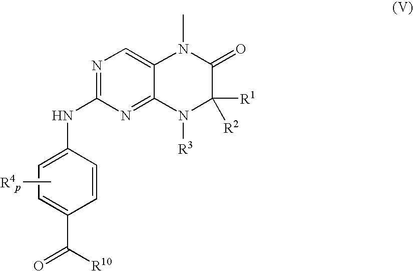

wherein R¹ to R⁴ is as hereinbefore defined; and R¹⁰ denotes OH, NH-Lₘ-R⁵ₙ, OMe or OEt, and
a) when R¹⁰ denotes NH-Lₘ-R⁵ₙ, reducing the compound of formula (V) to give a compound of formula (I1), or
b) when R¹⁰ denotes OH, OMe or OEt either
   i) optionally after previous hydrolysis of the ester group —COR¹⁰, reacting the compound of formula (V) with an amine of general formula (VI):

NH₂-Lₘ-R⁵ₙ (VI)
wherein R⁵ is as hereinbefore defined,
to give a compound of formula (Va)

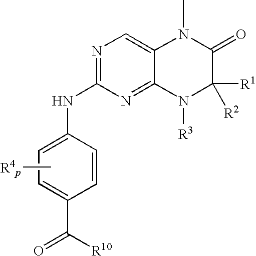

wherein R¹ to R⁴ is as hereinbefore defined; and R¹⁰ denotes NH-Lₘ-R⁵ₙ,
and reducing the compound of formula (Va) to give a compound of formula (II), or
ii) optionally after previous hydrolysis of the ester group —COR¹⁰, reducing the compound of formula (V) to give a compound of formula (VII)

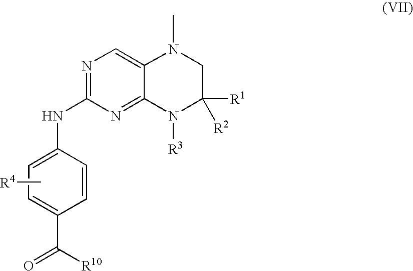

wherein R¹ to R⁴ is as hereinbefore defined; and R¹⁰ denotes OH, OMe or OEt,
and reacting the compound of formula (VII), optionally after previous hydrolysis of the ester group —COR¹⁰, with an amine of general formula (VI):

NH₂-Lₘ-R⁵ₙ (VI)
wherein R⁵ is as hereinbefore defined,
to give a compound of formula (II).

In one embodiment R¹⁰ is a substituent selected from among OH, NH₂-LR⁵, —O-methyl and —O-ethyl.

The term leaving group includes leaving groups such as for example —O-methyl, —SCN, chlorine, bromine, iodine, methanesulphonyl, trifluoromethanesulphonyl or p-toluenesulphonyl. In one embodiment the leaving group A is chlorine.

Methods for the preparation of compounds of Formula (III), (IV), (V) and (Va) are described in WO04/076454 and WO03/020722 and are incorporated herein by reference.

Compounds of Formula (III) may also be obtained by the cyclisation of a compound of formula (VIII)

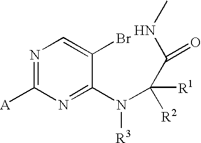

for example by palladium coupling.

Reducing agents suitable for the reduction of a compound of Formula (V) or (Va) include BH₃.SMe₂ and NaBH₄/BF₃.EtO₂.

It will be appreciated by those skilled in the art that in the processes of the present invention certain functional groups such as hydroxyl or amino groups in the starting reagents or intermediate compounds may need to be protected by protecting groups. Thus, the preparation of the compounds of formula (I) or (II) may involve, at various stages, the addition and removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 2nd edition, T. W. Greene and P. G. M. Wuts, Wiley Interscience (1991).

The invention further relates to compounds of formula (I) or (II) for use as pharmaceutical compositions.

In one embodiment of the invention, compounds of formula (I) or (II) are of use as pharmaceutical compositions with an antiproliferative activity.

The invention also relates to the use of a compound of formula (I) or (II) for preparing a pharmaceutical composition for the treatment and/or prevention of cancer, infections, inflammatory and autoimmune diseases.

These findings suggest that pharmacological inhibitors of Plk should be of therapeutic value for treatment of proliferative disease including solid tumours such as carcinomas and sarcomas and the leukaemias and lymphoid malignancies. In addition Plk inhibitors should be useful in the treatment of other disorders associated with uncontrolled cellular proliferation.

One aspect of the current invention therefore relates to the use of one or more of the compounds of formula (I) or (II) in the treatment of disorders characterised by excessive or anomalous cell proliferation.

Such diseases include for example: viral infections such as HIV and Kaposi's sarcoma; inflammatory and autoimmune diseases such as colitis, rheumatoid arthritis, Alzheimer's disease, glomerulonephritis and wound healing; bacterial, fungal and parasitic infections such as malaria and emphysema; dermatological diseases such as psoriasis; bone diseases; cardiovascular diseases such as restenosis and cardiomyopathy. The compounds in the present invention may be used for the prevention, short- or long-term treatment of the above-mentioned diseases, also in combination with other active substances used for the same indications.

The invention also relates to a method of treating and/or preventing cancer, infections, inflammatory and autoimmune diseases, characterised in that a patient is given an effective amount of a compound of formula (I) or (II).

The invention also relates to pharmaceutical preparations, containing as active substance one or more compounds of general formula (I) or (II), or the physiologically acceptable salts thereof, optionally combined with conventional excipients and/or carriers.

The compounds of formula (I) and (II) have activity as pharmaceuticals, in particular as modulators or inhibitors of Plk activity, and may be used in the treatment of proliferative and hyperproliferative diseases/conditions, examples of which include the following cancers:

(1) carcinoma, including that of the bladder, brain, breast, colon, kidney, liver, lung, ovary, pancreas, prostate, stomach, cervix, colon, thyroid and skin;

(2) hematopoietic tumours of lymphoid lineage, including acute lymphocytic leukaemia, B cell lymphoma and Burketts lymphoma;

(3) hematopoietic tumours of myeloid lineage, including acute and chronic myelogenous leukaemias and promyelocytic leukaemia;

(4) tumours of mesenchymal origin, including fibrosarcoma and rhabdomyosarcoma; and

(5) other tumours, including melanoma, seminoma, tetratocarcinoma, neuroblastoma and glioma.

In one embodiment, the compounds of formula (I) and (II) are useful in the treatment of tumours of the lung, breast and prostate.

Thus, the present invention provides a compound of formula (I) or (II), or a pharmaceutically acceptable salt thereof, as hereinbefore defined for use in therapy.

According to a further aspect of the present invention there is provided a compound of formula (I) or (II), or a pharmaceutically acceptable salt thereof, as hereinbefore defined for use in a method or treatment of the human or animal body by therapy.

In a further aspect, the present invention provides the use of a compound of formula (I) or (II), or a pharmaceutically acceptable salt thereof, as hereinbefore defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

The invention also provides a method of treating cancer which comprises administering to a patient in need thereof a therapeutically effective amount of a compound of formula (I) or (II), or a pharmaceutically acceptable salt thereof, as hereinbefore defined.

The invention still further provides a method of modulating polo-like kinase (Plk) activity which comprises administering to a patient in need thereof a therapeutically effective amount of a compound of formula (I) or (II), or a pharmaceutically acceptable salt thereof, as hereinbefore defined.

Thus according to this aspect of the invention there is provided a compound of the formula (I) or (II), or a pharmaceutically acceptable salt thereof, as defined herein for use as a medicament.

According to a further aspect of the invention there is provided the use of a compound of the formula (I) or (II), or a pharmaceutically acceptable salt thereof, as defined herein in the manufacture of a medicament for the production of a PLK inhibitory effect in a warm-blooded animal such as man.

According to this aspect of the invention there is provided the use of a compound of the formula (I) or (II), or a pharmaceutically acceptable salt thereof, as defined herein in the manufacture of a medicament for the production of an anti-cancer effect in a warm-blooded animal such as man.

According to a further feature of the invention, there is provided the use of a compound of the formula (I) or (II), or a pharmaceutically acceptable salt thereof, as defined herein in the manufacture of a medicament for the treatment of melanoma, papillary thyroid tumours, cholangiocarcinomas, colon cancer, ovarian cancer, lung cancer, leukaemias, lymphoid malignancies, multiple myeloma, carcinomas and sarcomas in the liver, kidney, bladder, prostate, breast and pancreas, and primary and recurrent solid tumours of the skin, colon, thyroid, lungs and ovaries.

According to a further aspect of the invention there is provided the use of a compound of the formula (I) or (II), or a pharmaceutically acceptable salt thereof, as defined herein in the production of a PLK inhibitory effect in a warm-blooded animal such as man.

According to this aspect of the invention there is provided the use of a compound of the formula (I) or (II), or a pharmaceutically acceptable salt thereof, as defined herein in the production of an anti-cancer effect in a warm-blooded animal such as man.

According to a further feature of the invention, there is provided the use of a compound of the formula (I) or (II), or a pharmaceutically acceptable salt thereof, as defined herein in the treatment of melanoma, papillary thyroid tumours, cholangiocarcinomas, colon cancer, ovarian cancer, lung cancer, leukaemias, lymphoid malignancies, multiple myeloma, carcinomas and sarcomas in the liver, kidney, bladder, prostate, breast and pancreas, and primary and recurrent solid tumours of the skin, colon, thyroid, lungs and ovaries.

According to a further feature of this aspect of the invention there is provided a method for producing a PLK inhibitory effect in a warm-blooded animal, such as man, in need of such treatment which comprises administering to said animal an effective amount of a compound of formula (I) or (II), or a pharmaceutically acceptable salt thereof, as defined herein.

According to a further feature of this aspect of the invention there is provided a method for producing an anti-cancer effect in a warm-blooded animal, such as man, in need of such treatment which comprises administering to said animal an effective amount of a compound of formula (I) or (II), or a pharmaceutically acceptable salt thereof, as defined herein.

According to an additional feature of this aspect of the invention there is provided a method of treating melanoma, papillary thyroid tumours, cholangiocarcinomas, colon cancer, ovarian cancer, lung cancer, leukaemias, lymphoid malignancies, multiple myeloma, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of a compound of formula (I) or (II)or a pharmaceutically acceptable salt thereof as defined herein.

In a further aspect of the invention there is provided a pharmaceutical composition which comprises a compound of the formula (I) or (II), or a pharmaceutically acceptable salt thereof, as defined herein in association with a pharmaceutically-acceptable diluent or carrier for use in the production of a PLK inhibitory effect in a warm-blooded animal such as man.

In a further aspect of the invention there is provided a pharmaceutical composition which comprises a compound of the formula (I) or (II), or a pharmaceutically acceptable salt thereof, as defined herein in association with a pharmaceutically-acceptable diluent or carrier for use in the production of an anti-cancer effect in a warm-blooded animal such as man.

In a further aspect of the invention there is provided a pharmaceutical composition which comprises a compound of the formula (I) or (II), or a pharmaceutically acceptable salt thereof, as defined herein in association with a pharmaceutically-acceptable diluent or carrier for use in the treatment of melanoma, papillary thyroid tumours, cholangiocarcinomas, colon cancer, ovarian cancer, lung cancer, leukaemias, lymphoid malignancies, multiple myeloma, carcinomas and sarcomas in the liver, kidney, bladder, prostate, breast and pancreas, and primary and recurrent solid tumours of the skin, colon, thyroid, lungs and ovaries in a warm-blooded animal such as man.

The compounds of formula (I) and (II), and pharmaceutically acceptable salts thereof, may be used on their own but will generally be administered in the form of a pharmaceutical composition in which the formula (I) or (II) compound or salt (active ingredient) is in association with a pharmaceutically acceptable adjuvant, diluent or carrier. Depending on the mode of administration, the pharmaceutical composition will preferably comprise from 0.05 to 99% w (per cent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I), or a pharmaceutically acceptable salt thereof, as hereinbefore defined, in association with a pharmaceutically acceptable adjuvant, diluent or carrier.

The invention further provides a process for the preparation of a pharmaceutical composition of the invention which comprises mixing a compound of formula (I) or (II), or a pharmaceutically acceptable salt thereof, as hereinbefore defined, with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane aerosols and dry powder formulations; or systemically, e.g. by oral administration in the form of tablets, capsules, syrups, powders or granules; or by parenteral administration in the form of solutions or suspensions; or by subcutaneous administration; or by rectal administration in the form of suppositories; or transdermally.

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate and anti oxidants such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p- hydroxybenzoate, anti oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil in water emulsions. The oily phase may be a vegetable oil, such as olive oil or arachis oil, or a mineral oil, such as for example liquid paraffin or a mixture of any of these. Suitable emulsifying agents may be, for example, naturally occurring gums such as gum acacia or gum tragacanth, naturally occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non toxic parenterally acceptable diluent or solvent, for example a solution in 1,3 butanediol.

Suppository formulations may be prepared by mixing the active ingredient with a suitable non irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedure well known in the art.

Compositions for administration by insufflation may be in the form of a finely divided powder containing particles of average diameter of, for example, 30μ or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo inhaler device, such as is used for insufflation of the known agent sodium cromoglycate.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The size of the dose for therapeutic purposes of a compound of the invention will naturally vary according to the nature and severity of the conditions, the age and sex of the animal or patient and the route of administration, according to well known principles of medicine.

In general, a compound of the invention will be administered so that a daily dose in the range, for example, from 0.5 mg to 75 mg active ingredient per kg body weight is received, given if required in divided doses. In general lower doses will be administered when a parenteral route is employed. Thus, for example, for intravenous administration, a dose in the range, for example, from 0.5 mg to 30 mg active ingredient per kg body weight will generally be used. Similarly, for administration by inhalation, a dose in the range, for example, from 0.5 mg to 25 mg active ingredient per kg body weight will generally be used. Also, for example, a formulation intended for oral administration to humans will generally contain, for example, from 0.5 mg to 2 g of active ingredient.

For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The anti cancer treatment defined hereinbefore may be applied as a sole therapy or may involve, in addition to the compound of the invention, conventional surgery or radiotherapy or chemotherapy. Such chemotherapy may include one or more of the following categories of anti-tumour agents:—

(i) other antiproliferative/antineoplastic drugs and combinations thereof, as used in medical oncology, such as alkylating agents (for example cisplatin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5 fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);

(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5*-reductase such as finasteride;

(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or antibodies to Heparanase);

(iv) inhibitors of growth factor function: for example such inhibitors include growth factor antibodies and growth factor receptor antibodies (for example the anti erbB2 antibody trastuzumab [Herceptin™], the anti-EGFR antibody panitumumab, the anti erbB1 antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI 774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)), inhibitors of cell signalling through MEK and/or AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD 1152, PH739358, VX-680, MLN8054, R763, MP235, MP529, VX-528 AND AX39459) and cyclin dependent kinase inhibitors such as CDK2 and/or CDK4 inhibitors;

(v) antiangiogenic agents such as those which inhibit the effects of vascular endothelial growth factor, [for example the anti vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), compounds such as those disclosed in International Patent Applications WO97/22596, WO 97/30035, WO 97/32856 and WO 98/13354 and compounds that work by other mechanisms (for example linomide, inhibitors of integrin avb3 function and angiostatin);

(vi) vascular damaging agents such as Combretastatin A4 and compounds disclosed in International Patent Applications WO 99/02166, WO 00/40529, WO 00/41669, WO 01/92224, WO 02/04434 and WO 02/08213;

(vii) antisense therapies, for example those which are directed to the targets listed above, such as ISIS 2503, an anti-ras antisense;

(viii) gene therapy approaches, including for example approaches to replace aberrant genes such as aberrant p53 or aberrant BRCA1 or BRCA2, GDEPT (gene directed enzyme pro drug therapy) approaches such as those using cytosine deaminase, thymidine kinase or a bacterial nitroreductase enzyme and approaches to increase patient tolerance to chemotherapy or radiotherapy such as multi drug resistance gene therapy;

(ix) immunotherapy approaches, including for example ex vivo and in vivo approaches to increase the immunogenicity of patient tumour cells, such as transfection with cytokines such as interleukin 2, interleukin 4 or granulocyte macrophage colony stimulating factor, approaches to decrease T cell anergy, approaches using transfected immune cells such as cytokine transfected dendritic cells, approaches using cytokine transfected tumour cell lines and approaches using anti idiotypic antibodies; and

(x) other inhibitors of cell cycle such as Eg5, Chk1 or PARP inhibitors.

### EXAMPLES

The invention will now be further described with reference to the following illustrative examples in which, unless stated otherwise:

(i) temperatures are given in degrees Celsius (° C.); operations were carried out at room or ambient temperature, that is, at a temperature in the range of 18-25° C.;

(ii) organic solutions were dried over anhydrous magnesium sulphate; evaporation of solvent was carried out using a rotary evaporator under reduced pressure (600-4000 Pascals; 4.5-30 mmHg) with a bath temperature of up to 60° C.;

(iii) chromatography means flash chromatography on silica gel;

(iv) SCX-2 cartridges are Ion Exchange SPE columns where the stationary phase is polymeric propylsulfonic acid. These are used to isolate amines.

(v) in general, the course of reactions was followed by TLC or LCMS and reaction times are given for illustration only;

(vi) final products had satisfactory proton nuclear magnetic resonance (NMR) spectra and/or mass spectral data;

(vii) yields are given for illustration only and are not necessarily those which can be obtained by diligent process development; preparations were repeated if more material was required;

(viii) when given, NMR data is in the form of delta values for major diagnostic protons, given in parts per million (ppm) relative to tetramethylsilane (TMS) as an internal standard, determined at 400 MHz or 500 MHz, in CDCl₃, DMSO-d₆ or DMSO-d6+d₄-AcOH unless otherwise indicated;

(ix) chemical symbols have their usual meanings; SI units and symbols are used;

(x) solvent ratios are given in volume:volume (v/v) terms; and

(xi) Mass spectra (MS) data was generated on an LCMS system where the HPLC component comprised generally either a Agilent 1100 or Waters Alliance HT (2790 & 2795) equipment and was run on a Phemonenex Gemini C18 5 mm, 50×2 mm column (or similar) eluting with either acidic eluent (for example, using a gradient between 0-95% water/acetonitrile with 5% of a 1% formic acid in 50:50 water:acetonitrile (v/v) mixture; or using an equivalent solvent system with methanol instead of acetonitrile), or basic eluent (for example, using a gradient between 0-95% water/acetonitrile with 5% of a 0.1% 880 Ammonia in acetonitrile mixture); and the MS component comprised generally a Waters ZQ mass spectrometer scanning over an appropriate mass range. Chromatograms for Electrospray (ESI) positive and negative Base Peak Intensity, and UV Total Absorption Chromatogram from 220-300 nm, are generated and values for m/z are given; generally, only ions which indicate the parent mass are reported and unless otherwise stated the value quoted is the (M+H)⁺ for positive ion mode and (M−H)− for negative ion mode;

(xii) the following abbreviations have been used:

| | |
|---|---|
| AcOH | acetic acid |
| d₄-AcOH | tetradeuteroacetic acid |
| CDCl₃ | deuterochloroform |
| DCM | dichloromethane |
| DIPEA | N,N-diisopropylethylamine |
| DMA | N,N-dimethylacetamide |
| DMF | N,N-dimethylformamide |
| DMSO | dimethylsulfoxide |
| DMSO-d₆ | hexadeuterodimethylsulfoxide |
| EtI | ethyl iodide |
| EtOH | ethanol |
| EtOAc | ethyl acetate |
| HATU | O-(7-azabenzotriazole-1-yl)-N,N,N′N′- |
| | tetramethyluronium hexafluorophosphate |
| HPLC | high performance liquid chromatography |
| MeCN | acetonitrile |
| MeOH | methanol |
| MeI | methyl iodide |
| MS | mass spectroscopy |
| m/z | mass to charge ratio |
| NMR | nuclear magnetic resonance |
| SCX-2 | ion exchange SPE column (polymeric propylsulfonic |
| | acid stationery phase) |
| THF | terahydrofuran |

### Example 1

### 4-[[(7R)-8-Cyclopentyl-7-ethyl-5-methyl-6,7-dihydropteridin-2-yl]amino]-3-methoxy-N-(1-methyl-4-piperidyl)benzamide

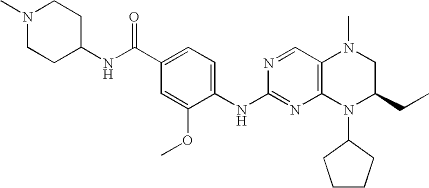

BH₃.SMe₂ (0.2 mL, 5.0 M in THF, 1 mmol) was added to solution of 4-[[(7R)-8-cyclopentyl-7-ethyl-5-methyl-6-oxo-7H-pteridin-2-yl]amino]-3-methoxy-N-(1-methyl-4-piperidyl)benzamide (WO2004076454; 53 mg, 0.1 mmol) in THF (20 mL) and stirred at ambient temperature for 3 h under an atmosphere of nitrogen. After 3 hours, HCl (20 mL, concentrated aqueous) was added and the resulting solution was stirred at ambient temperature for 16 h. The HCl solution was then diluted with water (200 mL), and loaded onto an SCX-2 column. The SCX-2 column was then washed with water (50 mL), then MeOH (50 mL). The crude product was then eluted from the column with NH₃ (50 mL, 7M in MeOH), and concentrated under reduced pressure. Purification by column chromatography (SiO₂, eluent gradient 0-10% NH₃ [7M in MeOH] in DCM) then by preparative HPLC (Xterra prep RP18, 19×100 mm column, eluting with a gradient composing of MeCN and a 1% solution of NH₃ in water), to give the title compound (29 mg, 57%) as a solid.

¹H NMR (400 MHz, CDCl₃): δ_{H} 0.94 (t, 3H), 1.50-1.83 (m, 10H), 1.91-1.95 (m, 1H), 2.08-2.11 (m, 3H), 2.36-2.38 (m, 2H), 2.43 (s, 3H), 2.74-2.77 (m, 1H), 2.80 (s, 3H), 3.01 (m, 2H,), 3.06-3.09 (m, 1H), 3.36-3.39 (m, 1H), 3.95 (s, 3H), 4.07 (m, 1H), 4.88 (m, 1H), 5.96 (d, 1H), 7.21-7.23 (m, 1H), 7.30 (s, 1H), 7.37 (d, 1H), 7.47 (s, 1H), 8.53 (d, 1H); MS m/z 508 [M+H]⁺.

### Example 2

### 4-[[(7R)-8-Cyclopentyl-7-ethyl-5-methyl-6,7-dihydropteridin-2-yl]amino]-N-(3-dimethylamino-2,2-dimethyl-propyl)-3-methoxy-benzamide

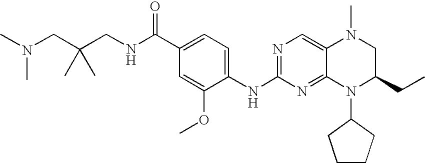

To a stirred solution of 4-[[(7R)-8-cyclopentyl-7-ethyl-5-methyl-6,7-dihydropteridin-2-yl]amino]-3-methoxy-benzoic acid (Intermediate 1; 30 mg, 0.07 mmol) in DMA (1 mL) was added N,N-2,2-tetramethyl-1,3-propanediamine (12 μL, 0.07 mmol) followed by DIPEA (64 μL, 0.36 mmol) and HATU (30 mg, 0.08 mmol) and the resulting solution stirred at ambient temperature for 1 h. The volatiles were removed under reduced pressure and the residue diluted with MeOH (10 mL) and loaded onto an SCX-2 column. The SCX-2 column was then washed with MeOH (10 mL). The crude product was then eluted from the SCX-2 column with NH₃ (10 mL, 7M in MeOH), and concentrated under reduced pressure. Purification by column chromatography (SiO₂, eluent gradient 5% NH₃ [3.5M in MeOH] in DCM) to afford the title compound (30 mg, 78%) as a solid.

¹H NMR (400 MHz, CDCl₃) δ_{H} 0.93 (t, 3H), 0.99 (s, 6H), 1.47-1.95 (m, 11H), 2.36 (s, 3H), 2.38 (s, 6H), 2.74 (ddd, 1H), 2.79 (s, 2H), 3.06 (dd, 1H), 3.33-3.39 (m, 2H), 3.95 (s, 3H), 4.87 (m, 1H), 7.26 (dd, 1H), 7.31 (s, 1H), 7.41 (s, 1H), 7.46 (d, 1H), 8.52 (d, 1H), 9.07 (t, 1H); MS m/z 524 [M+H]⁺.

### Example 3

### N-[[4-[[(7R)-8-Cyclopentyl-7-ethyl-5-methyl-6,7-dihydropteridin-2-yl]amino]-3-methoxy-phenyl]methyl]-N′,N′,2,2-tetramethyl-propane-1,3-diamine

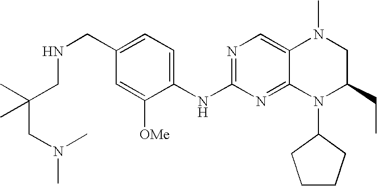

BH₃.SMe₂ (0.59 mL, 5.0 M in THF, 2.9 mmol) was added to solution of 4-[[(7R)-8-cyclopentyl-7-ethyl-5-methyl-6-oxo-7H-pteridin-2-yl]amino]-N-(3-dimethylamino-2,2-dimethyl-propyl)-3-methoxy-benzamide (WO2004076454; 159 mg, 0.29 mmol) in anhydrous THF (20 mL) and stirred at ambient temperature for 18 h under an atmosphere of nitrogen. Further BH₃.SMe₂ (0.59 mL, 5.0 M in THF, 2.9 mmol) was added and the reaction stirred at 45° C. for 1 h. HCl (20 mL, 2N aqueous) and MeOH (20 mL) were added and the volatiles removed under reduced pressure. The crude material was dissolved in MeOH (20 mL) and loaded onto a SCX-2 column. The SCX-2 column was then washed with MeOH (50 mL) and the crude product was eluted from the column with NH₃ (50 mL, 7M in MeOH) and the volatiles removed under reduced pressure. Purification by preparative HPLC (Gemini C18 5 um 30×100 mm column using a 35-95% MeCN gradient [Solvent A 99.5% water+0.5% NH₃, Solvent B 100% MeCN]) afforded the title compound (23 mg, 15%) as a gum.

¹H NMR (500 MHz, DMSO-d₆+d₄-AcOH) δ_{H} 0.85 (t, 3H), 0.95 (s, 6H), 1.42-1.70 (m, 8H), 1.80 (m, 1H), 1.90 (m, 1H), 2.43 (s, 6H), 2.69 (s, 6H), 2.73 (d, 1H), 2.81 (s, 3H), 3.15 (d, 1H), 3.52 (d, 1H), 3.81 (s, 3H), 4.00 (s, 2H), 4.51 (quintet, 1H), 6.98 (d. 1H), 7.06 (s, 1H), 7.11 (s, 1H), 7.92 (d, 1H); MS m/z 510 [M+H]⁺.

### Example 4

### (7R)-8-Cyclopentyl-7-ethyl-N-[3-fluoro-4-[[(1-methyl-4-piperidyl)amino]methyl]phenyl]-5-methyl-6,7-dihydropteridin-2-amine

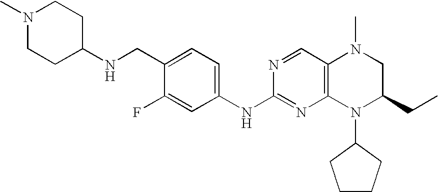

BH₃.SMe₂ (1.17 mL, 5.0 M in THF, 5.90 mmol) was added to a solution of 4-[[(7R)-8-cyclopentyl-7-ethyl-5-methyl-6-oxo-7H-pteridin-2-yl]amino]-2-fluoro-N-(1-methyl-4-piperidyl)benzamide (Intermediate 4; 301 mg, 0.59 mmol) in THF (20 mL) and stirred for 18 h at ambient temperature under an atmosphere of nitrogen. Further BH₃.SMe₂ (0.58 mL, 5.0 M in THF, 2.95 mmol) was added and the reaction stirred at ambient temperature for 1 h. HCl (20 mL, 2N aqueous) and MeOH (20 mL) were added and the volatiles removed under reduced pressure. The crude material was dissolved in MeOH (20 mL) and loaded onto a SCX-2 column. The SCX-2 column was then washed with MeOH (50 mL) and the crude product was eluted from the column with NH₃ (50 mL, 7M in MeOH) and the volatiles removed under reduced pressure. Purification by preparative HPLC (Gemini C18 5 um 30×100 mm column, 35-95% MeCN gradient) afforded the desired compound as the mono-borane complex. The borane complex was heated at 50° C. in THF (10 mL), MeOH (10 mL) and HCl (10 mL, concentrated aqueous) for 2 h. The reaction mixture was cooled and loaded onto an SCX-2 column then washed with methanol (20 mL). The crude compound was eluted from the SCX-2 column with NH₃ (40 mL, 7M in methanol). Purification by column chromatography (SiO₂, gradient eluent: 2-10% MeOH in DCM) afforded the title compound (30 mg, 11%) as a white solid.

¹H NMR (400 MHz, DMSO-d₆): δ_{H} 0.91 (t, 3H), 1.28 (m, 2H), 1.45-1.90 (m, 11H), 1.97 (m, 1H), 2.13 (s, 3H), 2.32 (m, 1H), 2.60 (d, 1H), 2.68 (d, 2H), 2.75 (s, 3H), 3.15 9d, 1H), 3.29 (s, 3H), 3.46 (d, 1H), 3.64 (s, 2H), 4.82 (quintet, 1H), 7.20 (tr, 1H), 7.28 (m, 1H), 7.34 (s, 1H), 7.77 (d, 1H), 8.73 (s, 1H); MS m/z 482 [M+H]⁺.

### Example 5

### 4-[[(7R)-8-Cyclopentyl-5,7-diethyl-6,7-dihydropteridin-2-yl]amino]-3-methoxy-N-(1-methyl-4-piperidyl)benzamide

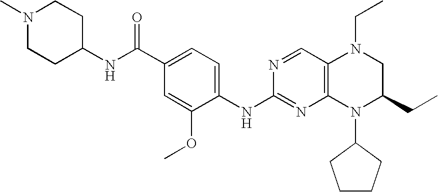

To a solution of 4-{[(7R)-8-cyclopentyl-5,7-diethyl-5,6,7,8-tetrahydropteridin-2-yl]amino}-3-methoxybenzoic acid (Intermediate 7; 50 mg, 0.12 mmol) and N-[(1H-1,2,3-benzotriazol-1-yloxy)(dimethylamino)methylene]-N-methylmethanaminium tetrafluoroborate (47 mg, 0.12 mmol) in DMF (5 mL) was added 1-methylpiperidin-4-amine (17 uL, 0.12 mmol) and N-ethyl-N-isopropylpropan-2-amine (66 uL, 0.38 mmol), and the reaction stirred at ambient temperature for 2 h. The volatiles were removed under reduced pressure and the crude material was dissolved in DCM (10 mL), washed with NaOH (2.0M aqueous, 10 mL), dried (MgSO₄) and filtered. The volatiles were then removed under reduced pressure. Purification by column chromatography (SiO₂, eluent gradient 0-10% MeOH in DCM then 5% NH₃ [3.5M in MeOH] in DCM) afforded the title compound (31 mg, 50%) as a solid.

¹H NMR (400 MHz, DMSO-d₆) δ_{H} 0.91 (t, 3H), 1.09 (t, 3H), 1.71 (m, 8H), 2.71 (m, 4H), 2.79 (s, 3H), 2.95 (s, 3H), 3.21 (m, 4H), 3.51 (m, 1H), 3.90 (m, 4H), 3.94 (s, 3H), 4.75 (m, 1H), 7.25 (s, 1H), 7.38 (s, 1H), 7.44 (s, 1H), 7.46 (d, 1H), 8.09 (d, 1H), 8.47 (d, 1H); MS m/z 523 [M+H]⁺.

### Example 6

### 4-{[(7R)-8-Cyclopentyl-7-cyclopropyl-5-methyl-5,6,7,8-tetrahydropteridin-2-yl]amino}-3-methoxy-N-(1-methylpiperidin-4-yl)benzamide

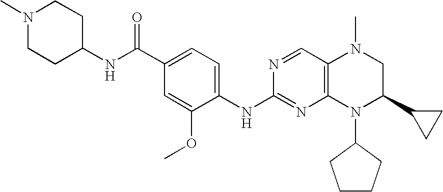

To a solution of 4-{[(7R)-8-cyclopentyl-7-cyclopropyl-5-methyl-5,6,7,8-tetrahydropteridin-2-yl]amino}-3-methoxybenzoic acid (Intermediate 11; 50 mg, 0.12 mmol) and N-[(1H-1,2,3-benzotriazol-1-yloxy)(dimethylamino)methylene]-N-methylmethanaminium tetrafluoroborate (47 mg, 0.12 mmol) in DMF (5 mL) was added 1-methylpiperidin-4-amine (17 uL, 0.12 mmol) and DIPEA (66 uL, 0.38 mmol), and the reaction stirred at ambient temperature for 2 h. The volatiles were removed under reduced pressure and the crude material was re-dissolved in DCM (10 mL), washed with NaOH (10 mL, 2.0M aqueous), dried (MgSO₄) and filtered. The volatiles were then removed under reduced pressure. Purification by column chromatography (SiO₂, eluent gradient 0-10% MeOH in DCM then 5% NH₃ (3.5M in MeOH) in DCM) afforded the title compound (18 mg, 29%) as a solid.

¹H NMR (400 MHz, DMSO-d₆) δ_{H} 0.31 (m, 1H), 0.55 (m, 2H), 1.00 (m, 1H), 1.61 (m, 4H), 1.83 (m, 7H), 2.77 (s, 3H), 2.83 (m, 2H), 2.90 (s, 3H), 3.11 (m, 8H), 3.87 (s, 3H), 4.53 (m, 1H), 7.26 (s, 1H), 7.36 (s, 1H), 7.46 (s, 2H), 8.11 (d, 1H), 8.48 (d, 1H); MS m/z 520 [M+H]⁺.

### Example 7

### 4-{[(7R)-8-Cyclopentyl-5-methyl-7-propyl-5,6,7,8-tetrahydropteridin-2-yl]amino}-3-methoxy-N-(1-methylpiperidin-4-yl)benzamide

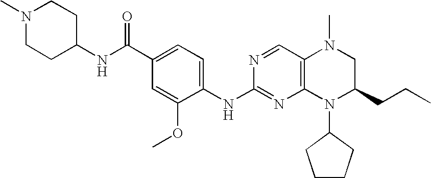

To a solution of 4-{[(7R)-8-cyclopentyl-5-methyl-7-propyl-5,6,7,8-tetrahydropteridin-2-yl]amino}-3-methoxybenzoic acid (Intermediate 18; 30 mg, 0.07 mmol) and N-[(1H-1,2,3-benzotriazol-1-yloxy)(dimethylamino)methylene]-N-methylmethanaminium tetrafluoroborate (29 mg, 0.07 mmol) in DMF (5 mL) was added 1-methylpiperidin-4-amine (11 uL, 0.07 mmol) and DIPEA (40 uL, 0.23 mmol), and the reaction stirred at ambient temperature for 2 h. The volatiles were removed under reduced pressure and the crude material was re-dissolved in DCM (10 mL), washed with NaOH (2.0M aqueous, 10 mL), dried (MgSO4) and filtered. The volatiles were then removed under reduced pressure. Purification by column chromatography (SiO₂, eluent gradient 0-10% MeOH in DCM then 5% NH₃ [3.5M in MeOH] in DCM) afforded the title compound (27 mg, 74%) as a solid.

¹H NMR (400 MHz, DMSO-d₆) δ_{H} 0.91 (t, 3H), 1.43 (m, 2H), 1.77 (m, 3H), 2.05 (m, 6H), 2.70 (m, 7H), 2.77 (s, 3H), 3.02 (t, 3H), 3.12 (d, 2H), 3.34 (m, 3H), 3.59 (m, 1H), 3.93 (s, 3H), 4.02 (m, 1H), 4.68 (m, 1H), 7.24 (s, 1H), 7.41 (s, 1H), 7.43 (s, 1H), 7.92 (s, 1H), 8.33 (d, 1H); MS m/z 523 [M+H]⁺.

### Example 8

### 4-[[(7R)-8-Cyclopentyl-7-ethyl-5-methyl-6,7-dihydropteridin-2-yl]amino]-3-ethoxy-N-(1-methyl-4-piperidyl)benzamide

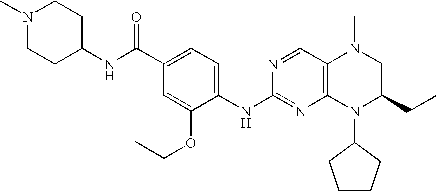

BH₃.SMe₂ (0.405 mL, 5.0 M in THF, 1 mmol) was added to solution of 4-[[(7R)-8-cyclopentyl-7-ethyl-5-methyl-6-oxo-7H-pteridin-2-yl]amino]-3 -ethoxy-N-(1-methyl-4-piperidyl)benzamide (Intermediate 19; 107 mg, 0.2 mmol) in THF (20 mL) and stirred at ambient temperature for 2 h under an atmosphere of nitrogen. After 2 h, HCl (20 mL, concentrated aqueous) was added and the resulting solution was stirred at ambient temperature for 1 h. The HCl solution was then diluted with water (20 mL), and loaded onto an SCX-2 column. The SCX-2 column was then washed with water (50 mL), then MeOH (50 mL). The crude product was then eluted from the column with NH₃ (50 mL, 7M in MeOH), and concentrated under reduced pressure. Purification by column chromatography (SiO₂, eluent gradient 0-10% NH₃ (7M in MeOH) in DCM) then by preparative HPLC (Xterra prep RP 18, 19×100 mm column, eluting with a gradient composing of MeCN and a 1% solution of NH₃ in water), to give the title compound (3 mg, 3%) as a solid.

¹H NMR (400 MHz, CDCl₃) δ0.79 (t, 3H), 1.34 (t, 3H), 1.45-1.82 (m, 10H), 1.89- 1.99 (m, 4H), 2.04 (t, 2H), 2.17 (s, 3H), 2.55-2.63 (m, 1H), 2.63 (s, 3H), 2.93 (d, 1H), 3.01 (d, 1H), 3.22 (d, 1H), 3.84-3.89 (m, 2H), 4.04 (q, 2H), 4.67-4.71 (m, 1H), 5.73-5.76 (m, 1H), 7.05 (d, 1H), 7.17 (s, 1H), 7.22 (s, 1H), 7.28 (s, 1H), 8.38 (d, 1H); MS m/z 521 [M+H]⁺.

### Intermediates

### Intermediate 1

### 4-[[(7R)-S-Cyclopentyl-7-ethyl-5-methyl-6,7-dihydropteridin-2-yl]amino]-3-methoxy-benzoic acid

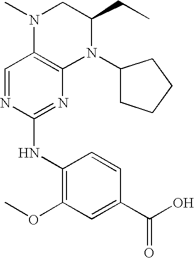

4-Methylpentan-(2R/S)-yl 4-[[(7R)-8-cyclopentyl-7-ethyl-5-methyl-6,7-dihydropteridin-2-yl]amino]-3-methoxy-benzoate (Intermediate 2; 100 mg, 0.20 mmol) was suspended in water (2 mL), treated with HCl (1 mL, concentrated aqueous) and heated at reflux for 5 h. The reaction mixture was cooled and the volatiles removed under reduced pressure. Purification by preparative HPLC (Xterra prep RP18, 19×100 mm column, eluting with a gradient composing of MeCN and a 1% solution of NH₃ in water) to give the title compound (31 mg, 38%) as a solid.

¹H NMR (400 MHz, DMSO-d₆) δ_{H} 0.92 (t, 3H), 1.47-1.86 (m, 9H), 1.93- 2.02 (m, 1H), 2.64-2.70 (m, 1H), 2.78 (s, 3H), 3.17 (d, 1H), 3.49 (d, 1H), 3.94 (s, 3H), 4.74 (quintet, 1H), 7.35 (m, 2H), 7.46 (s, 1H), 7.54 (d, 1H), 8.55 (d, 1H), 12.46 (br s, 1H); MS m/z 412 [M+H]⁺.

### Intermediate 2

### 4-Methylpentan-(2R/S)-yl 4-[[(7R)-8-cyclopentyl-7-ethyl-5-methyl-6,7-dihydropteridin-2-yl]amino]-3-methoxy-benzoate

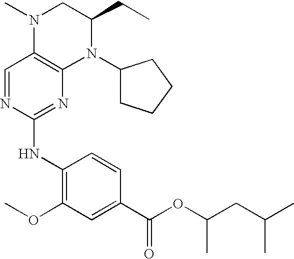

BH₃.SMe₂ (0.73 mL, 5.0 M in THF, 3.7 mmol) was added to solution of 4-methylpentan-(2R/S)-yl 4-[[(7R)-8-cyclopentyl-7-ethyl-5-methyl-6-oxo-7H-pteridin-2-yl]amino]-3-methoxy-benzoate (Intermediate 3; 185 mg, 0.36 mmol) in THF (20 mL) and stirred at ambient temperature for 5 h under an atmosphere of nitrogen. After 5 hours, HCl (5 mL, concentrated aqueous) was added and the resulting solution was stirred at ambient temperature for 16 h. The HCl solution was then diluted with water (200 mL), and loaded onto an SCX-2 column. The SCX-2 column was then washed with water (100 mL), then MeOH (100 mL). The crude product was then eluted from the SCX-2 column with NH₃ (100 mL, 7M in MeOH), and concentrated under reduced pressure. Purification by column chromatography (SiO₂, eluent gradient 0-40% EtOAc in iso-hexane) to afford the title compound (116 mg, 65%) as a solid.

¹H NMR (40002 MHz, DMSO-d₆) δ_{H} 0.89-0.95 (m, 9H), 1.29 (d, 3H), 1.37 -1.48 (m, 1H), 1.48-1.89 (m, 10H), 1.93-2.02 (m, 1H), 2.67 (dd, 1H), 2.78 (s, 3H), 3.17 (dd, 1H), 3.45-3.53 (m, 1H), 3.95 (s, 3H), 4.74 (quintet, 1H), 5.08-5.16 (m, 1H), 7.35 (s, 1H), 7.38 (s, 1H), 7.45 (d, 1H), 7.55 (dd, 1H), 8.56 (d, 1H); MS m/z 496 [M+H]⁺.

### Intermediate 3

### 4-Methylpentan-(2R/S)-yl 4-[[(7R)-8-cyclopentyl-7-ethyl-5-methyl-6-oxo-7H-pteridin-2-yl]amino]-3-methoxy-benzoate

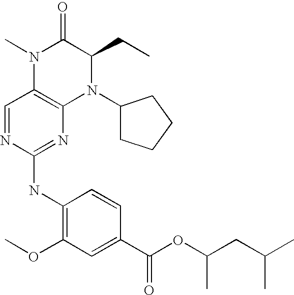

(7R)-2-Chloro-8-cyclopentyl-7-ethyl-5-methyl-7H-pteridin-6-one (WO2004076454; 200 mg, 0.68 mmol), methyl 4-amino-3-methoxy-benzoate (123 mg, 0.68 mmol) and p-toluenesulfonic acid (323 mg, 1.70 mmol) were suspended in (2R/S)-4-methyl-2-pentanol (1 mL) and heated at reflux for 5 h, allowing the MeOH evaporate during the reaction. The reaction mixture was cooled and loaded onto an SCX-2 column and washed with MeOH (40 mL). The crude product was then eluted from the SCX-2 column with NH₃(40 mL, 7M in MeOH) and the volatiles removed under reduced pressure. Purification by column chromatography (SiO₂, eluent gradient 0-100% EtOAc in iso-hexane) afforded the title compound (185 mg, 53%) as a gum.

¹H NMR (400 MHz, DMSO-d₆) δ_{H} 0.77 (t, 3H), 0.91 (dd, 6H), 1.29 (d, 3H), 1.37-1.48 (m, 1H), 1.55-2.08 (m, 12H), 3.30 (s, 3H), 3.95 (s, 3H), 4.25 (dd, 1H), 4.37 (quintet, 1H), 5.09-5.17 (m, 1H), 7.49 (d, 1H), 7.58 (dd, 1H), 7.71 (s, 1H), 7.86 (s, 1H), 8.51 (dt, 1H); MS m/z 510 [M+H]⁺.

### Intermediate 4

### 4-[[(7R)-8-Cyclopentyl-7-ethyl-5-methyl-6-oxo-7H-pteridin-2-yl]amino]-2-fluoro-N-(1-methyl-4-piperidyl)benzamide

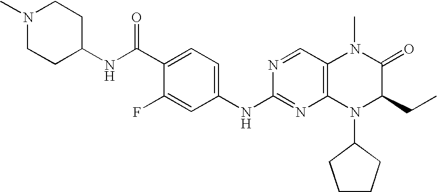

(7R)-2-chloro-8-cyclopentyl-7-ethyl-5-methyl-7H-pteridin-6-one (WO2004076454; 281 mg, 0.95 mmol)), 4-amino-2-fluoro-N-(1-methyl-4-piperidyl)benzamide (Intermediate 5; 263 mg, 1.05 mmol) and p-toluenesulfonic acid (452 mg, 2.38 mmol) were suspended in (2R/S)-4-methyl-2-pentanol (10 mL) and heated at reflux for 12 h. The reaction mixture was cooled and loaded onto an SCX-2 column then washed with MeOH (20 mL). The crude compound was eluted from the SCX-2 column with NH₃ (40 mL, 7M in MeOH). Purification by column chromatography (SiO₂, eluting with MeOH) afforded a gum. The gum was dissolved in DCM and filtered to afford the title compound (319 mg, 66%) as a brown solid.

¹H NMR (400 MHz, DMSO-d₆) δ_{H} 0.75 (tr, 3H), 1.60-2.10 (m, 14H), 2.74 (s, 3H), 3.09 (m, 2H), 3.22 (s, 3H), 3.39 (m, 2H), 4.03 (m, 1H), 4.20 (m, 1H), 4.41 (s, 1H), 7.38 (d, 1H), 7.53 (tr, 1H), 7.78 (s, 1H), 7.89 (d, 1H); MS m/z 510 [M+H]⁺.

### Intermediate 5

### 4-Amino-2-fluoro-N-(1-methyl-4-piperidyl)benzamide

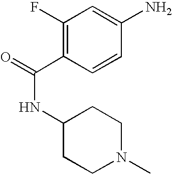

A suspension of 2-fluoro-N-(1-methyl-4-piperidyl)-4-nitro-benzamide (Intermediate 6; 5.95 g, 21.1 mmol) and Pt (595 mg, 5% on charcoal) in EtOH (300 mL) was stirred at 40° C. under a hydrogen atmosphere for 16 h. The reaction mixture was then filtered through celite, washed with MeOH (100 mL) and the volatiles removed under reduced pressure to afforded the title compound (5.04 g, 95%) as a yellow foam.

¹H NMR (400 MHz, DMSO-d₆) δ_{H} 1.60 (m, 2H), 1.81 (m, 2H), 2.05 (tr, 2H), 2.22 (s, 3H), 2.78 (m, 2H), 3.77 (m, 1H), 5.93 (s, 2H), 6.36 (dd, 1H), 6.45 (d, 1H), 7.45(m, 2H); MS m/z 252 [M+H]⁺.

### Intermediate 6

### 2-Fluoro-N-(1-methyl-4-piperidyl)-4-nitro-benzamide

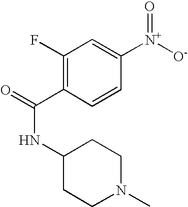

To a suspension of 2-fluoro-4-nitro-benzoic acid (5 g, 27.0 mmol), 4-amino-1-methylpiperidine (3.4 g, 29.7 mmol) and DIPEA (9.39 mL, 54.0 mmol) in anhydrous DMA (100 mL) under nitrogen was added HATU (11.3 g, 29.7 mmol) and the resulting reaction mixture stirred at ambient temperature for 4 h. The volatiles were then removed under reduced pressure and the resulting residue was dissolved in DCM (100 mL). The organic phase was washed with NaHCO₃ (100 mL, sat. aq.) and brine (100 mL). Purification by column chromatography (SiO₂, gradient eluent 2-30% MeOH in DCM) afforded the title compound (5.95 g, 78%) as a yellow solid.

¹H NMR (400 MHz, DMSO-D6) δ_{H} 1.57 (m, 2H), 1.83 (m, 2H), 2.07 (tr, 2H), 2.21 (s, 3H), 2.78 (m, 1H), 3.72 (m, 1H), 7.79 (tr, 1H), 8.13 (m, 1H), 8.19 (m, 1H), 8.58 (d, 1H); MS m/z 282 [M+H]⁺.

### Intermediate 7

### 4-{[(7R)-⁸-Cyclopentyl-5,7-diethyl-5,6,7,8-tetrahydropteridin-2-yl]amino}-3-methoxybenzoic acid

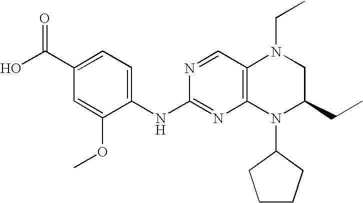

Methyl 4-{[(7R)-8-cyclopentyl-5,7-diethyl-5,6,7,8-tetrahydropteridin-2-yl]amino}-3-methoxybenzoate (Intermediate 8; 120 mg, 0.27 mmol) and HCl (1 mL, concentrated aqueous) were suspended in water (2 mL) and heated at reflux for 24 h. The reaction mixture was then cooled to ambient temperature and the volatiles were removed under reduced pressure. Purification by preparative HPLC (Gemini C18 5 um 30×100 mm column, gradient eluent: 35-55% MeCN [Solvent A 99.5% water+0.5% NH₃, Solvent B 100% MeCN]) afforded the title compound (55 mg, 44%) as a solid.

¹H NMR (400.1 MHz, DMSO-d₆) δ_{H} 0.85 (t, 3H), 1.04 (t, 3H), 1.67 (m, 5H), 1.96 (m, 3H), 2.73 (d, 2H), 3.31 (m, 4H), 3.50 (d, 1H), 3.93 (s, 3H), 4.74 (m, 1H), 7.31 (s, 1H), 7.39 (s, 1H), 7.45 (s, 1H), 7.53 (d, 1H), 8.54 (d, 1H); MS m/z 427 [M+H]⁺.

### Intermediate 8

### Methyl 4-{[(7R)-8-cyclopentyl-5,7-diethyl-5,6,7,8-tetrahydropteridin-2-yl]amino}-3-methoxybenzoate

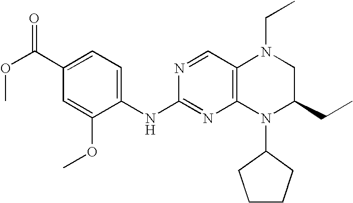

BH₃.SMe₂ (0.58 mL, 5.0 M in diethyl ether, 4.3 mmol) was added to a solution of methyl 4-{[(7R)-8-cyclopentyl-5,7-diethyl-6-oxo-5,6,7,8-tetrahydropteridin-2-yl]amino}-3-methoxybenzoate (Intermediate 9; 130 mg, 0.29 mmol) in THF (4 mL) and stirred for 5 h at ambient temperature under an atmosphere of nitrogen. HCl (10 mL, concentrated aqueous) was added and the resulting solution was stirred at ambient temperature for 16 h. The HCl solution was then diluted with water (50 mL), and loaded onto an SCX-2 column. The SCX-2 column was then washed with water (50 mL) and MeOH (50 mL). The product was then eluted from the SCX-2 column with NH₃ (50 mL, 7M in MeOH). The volatiles were then removed under reduced pressure to afford the title compound (120 mg, 84%) as a solid.

¹H NMR (400 MHz, DMSO-d₆) δ_{H} 0.85 (t, 3H), 1.04 (t, 3H), 1.68 (m, 11H), 1.96 (m, 1H), 2.74 (m, 1H), 3.19 (m, 1H), 3.51 (m, 1H), 3.82 (s, 3H), 3.95 (s, 3H), 4.74 (quintet, 1H), 7.36 (s, 1H), 7.39 (s, 1H), 7.46 (d, 1H), 7.56 (m, 1H), 8.58 (d, 1H); MS m/z 441 [M+H]⁺.

### Intermediate 9

### Methyl 4-{[(7R)-8-cyclopentyl-5,7-diethyl-6-oxo-5,6,7,8-tetrahydropteridin-2-yl]amino}-3-methoxybenzoate

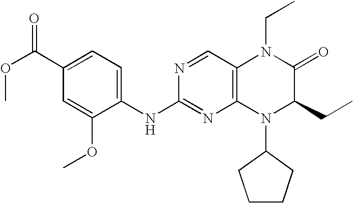

((7R)-2-chloro-8-cyclopentyl-5,7-diethyl-7,8-dihydropteridin-6(5H)-one (350 mg, 1.14 mmol), methyl 4-amino-3-methoxybenzoate (Intermediate 10; 208 mg, 1.14 mmol) and p-toluenesulfonic acid (543 mg, 2.85 mmol) were suspended in (2R/S)-4-methyl-2-pentanol (10 mL) and heated at reflux for 5 h. The reaction mixture was cooled and loaded onto an SCX-2 column and washed with MeOH (20 mL). The crude product was then eluted from the SCX-2 column with NH₃ (40 mL, 7M in MeOH) and the volatiles removed under reduced pressure. Purification by column chromatography (SiO₂, eluent gradient 0-100% EtOAc in iso-hexane) afforded the title compound (130 mg, 25%) as a solid.

¹H NMR (400 MHz, DMSO-D6) δ_{H} 0.78 (t, 3H), 1.14 (t, 3H), 1.64 (m, 3H), 1.83 (m, 6H), 2.05 (m, 1H), 3.78 (m, 1H), 3.84 (s, 3H), 3.97 (s, 3H), 4.02 (m, 1H), 4.23 (m, 1H), 4.38 (quintet, 1H), 7.52 (d, 1H), 7.61 (m, 1H), 7.72 (s, 1H), 7.93 (s, 1H), 8.55 (d, 1H); MS m/z 455 [M+H]⁺.

### Intermediate 10

### (7R)-2-Chloro-8-cyclopentyl-5,7-diethyl-7H-pteridin-6-one

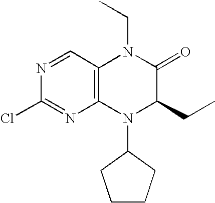

To a cold (0° C.) solution of (7R)-2-Chloro-8-cyclopentyl-7-ethyl-5,7-dihydropteridin-6-one (WO2004076454 ; 500 mg, 1.77 mmol) and EtI (156 uL, 1.95 mmol) in DMA (5 mL) was added NaH (76 mg, 1.89 mmol) and the reaction mixture stirred at 0° C. for 30 mins, then at ambient temperature for 1 h. Water (10 mL) was added and the volatiles were removed under reduced pressure. Purification by column chromatography (SiO₂, eluent gradient: 0-10% MeOH in DCM) afforded the title compound (300 mg, 55%) as a solid.

¹H NMR (400 MHz, DMSO-d₆) δ_{H} 0.74 (t, 3H), 1.13 (t, 3H), 1.57 (m, 2H), 1.69 (m, 2H), 1.85 (m, 6H), 3.76 (sextet, 1H), 3.99 (sextet, 1H), 4.17 (quintet, 1H), 4.32 (m, 1H), 7.94 (s, 1H); MS m/z 310 [M+H]⁺.

### Intermediate 11

### 4-{[(7R)-8-Cyclopentyl-7-cyclopropyl-5-methyl-5,6,7,8-tetrahydropteridin-2-yl]amino}-3-methoxybenzoic acid

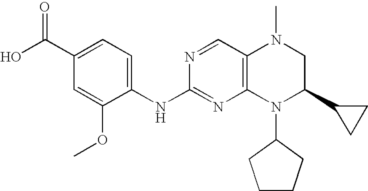

Methyl 4-{[(7R)-8-cyclopentyl-7-cyclopropyl-5 -methyl-5,6,7,8-tetrahydropteridin-2-yl]amino}-3-methoxybenzoate (Intermediate 12; 120 mg, 0.27 mmol) and HCl (1 mL, concentrated aqueous) were suspended in water (2 mL) and heated at reflux for 24 h. The reaction mixture was cooled to ambient temperature and the volatiles were removed under reduced pressure. Purification by preparative HPLC (Gemini C18 5 um 30×100 mm column, eluent gradient: 35-55% MeCN gradient [Solvent A 99.5% water+0.5% NH₃, Solvent B 100% MeCN]) afforded the title compound (55 mg, 44%) as a solid.

¹H NMR (400MHz, DMSO-d₆) δ_{H} 0.36 (m, 1H), 0.53 (m, 1H), 0.65 (m, 2H), 1.04 (m, 1H), 1.55 (m, 5H), 1.85 (m, 5H), 2.79 (s, 3H), 3.00 (m, 1H), 3.23 (m, 1H), 3.92 (s, 3H), 4.41 (m, 1H), 7.28 (s, 1H), 7.59 (s, 1H), 7.98 (d, 1H), 9.39 (s, 1H), 12.76 (s, 1H); MS m/z 424.5 [M+H]⁺.

### Intermediate 12

### Methyl 4-{[(7R)-8-cyclopentyl-7-cyclopropyl-5-methyl-5,6,7,8-tetrahydropteridin-2-yl]amino}-3-methoxybenzoate

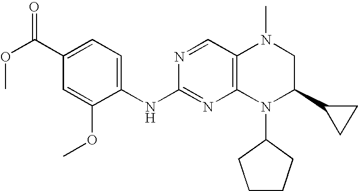

BH₃.SMe₂ (0.78 mL, 5.0 M in diethyl ether, 3.76 mmol) was added to a solution of methyl 4-{[(7R)-8-cyclopentyl-7-cyclopropyl-5 -methyl-6-oxo-5,6,7,8-tetrahydropteridin-2-yl]amino}-3-methoxybenzoate (Intermediate 13; 170 mg, 0.38 mmol) in THF (4 mL) and stirred for 3 h at ambient temperature under an atmosphere of nitrogen. HCl (10 mL, concentrated aqueous) was added and the resulting solution was stirred at ambient temperature for 16 h. The HCl solution was then diluted with water (50 mL), and loaded onto an SCX-2 column. The SCX-2 column was then washed with water (50 mL) and MeOH (50 mL). The product was then eluted from the column with NH₃ (50 mL, 7M in MeOH). The volatiles were then removed under reduced pressure to afford the title compound (120 mg, 72%) as a solid.

¹H NMR (400.1 MHz, DMSO-d₆) δ_{H} 0.31 (m, 1H), 0.55 (m, 4H), 1.62 (m, 2H), 1.79 (m, 4H), 1.95 (m, 2H), 2.78 (s, 3H), 2.85 (m, 1H), 3.12 (m, 2H), 3.82 (s, 3H), 3.95 (s, 3H), 4.52 (quintet, 1H), 7.37 (s, 1H), 7.41 (s, 1H), 7.46 (d, 1H), 7.57 (m, 1H), 8.57 (d, 1H); MS m/z 438.5 [M+H]⁺.

### Intermediate 13

### Methyl 4-{[(7R)-8-cyclopentyl-7-cyclopropyl-5-methyl-6-oxo-5,6,7,8-tetrahydropteridin-2-yl]amino}-3-methoxybenzoate

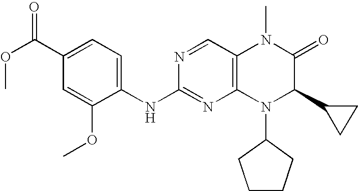

(7R)-2-chloro-8-cyclopentyl-7-cyclopropyl-5-methyl-7,8-dihydropteridin-6(5H)-one (Intermediate 14; 350 mg, 1.14 mmol), methyl 4-amino-3-methoxybenzoate (208 mg, 1.14 mmol) and p-toluenesulfonic acid (543 mg, 2.85 mmol) were suspended in (2R/S)-4-methyl-2-pentanol (10 mL) and heated at reflux for 5 h. The reaction mixture was cooled and loaded onto an SCX-2 column and washed with MeOH (20 mL). The crude product was then eluted from the SCX-2 column with NH₃ (40 mL, 7M in MeOH) and the volatiles removed under reduced pressure. Purification by column chromatography (SiO₂, eluent gradient 0-100% EtOAc in iso-hexane) afforded the title compound (170 mg, 33%) as a solid.

¹H NMR (400 MHz, DMSO-d₆) δ_{H} 0.50 (m, 4H), 0.92 (m, 1H), 1.62 (m, 2H), 1.80 (m, 2H), 1.95 (m, 3H), 2.06 (m, 1H), 3.27 (s, 3H), 3.76 (d, 1H), 3.84 (s, 3H), 3.97 (s, 3H), 4.36 (quintet, 1H), 7.51 (d, 1H), 7.61 (m, 1H), 7.75 (s, 1H), 7.95 (s, 1H), 8.55 (d, 1H); MS m/z 454.5 [M+H]⁺.

### Intermediate 14

### (7R)-2-Chloro-8-cyclopentyl-7-cyclopropyl-S-methyl-7,8-dihydropteridin-6(5H)-one

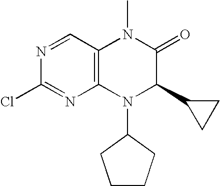

To a cold (0° C.) solution of (7R)-2-chloro-8-cyclopentyl-7-cyclopropyl-7,8-dihydropteridin-6(5h)-one (Intermediate 15; 470 mg, 1.60 mmol) and MeI (110 uL, 1.76 mmol) in DMA (5 mL) was added NaH (117 mg, 1.70 mmol) and the reaction stirred at 0° C. for 30 mins, then at ambient temperature for 1 h. Water (10 mL) was added which resulted in the precipitation of a solid. This was filtered to afford the title compound (370 mg, 76%) as a solid.

¹H NMR (400 MHz, DMSO-d₆) δ_{H} 0.46 (m, 2H), 0.57 (m, 2H), 0.94 (m, 1H), 1.56 (m, 2H), 1.88 (m, 6H), 3.25 (s, 3H), 3.79 (d, 1H), 4.20 (quintet, 1H), 7.95 (s, 1H); MS m/z 307 [M+H]⁺.

### Intermediate 15

### (7R)-2-Chloro-8-cyclopentyl-7-cyclopropyl-5-methyl-7,8-dihydropteridin-6(5H)-one

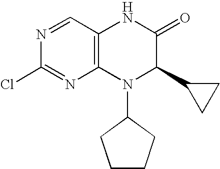

To a solution of methyl(2R)-[(2-chloro-5-nitropyrimidin-4-yl)(cyclopentyl)amino]-(cyclopropyl)acetate (Intermediate 16; 1 g, 2.80 mmol) in AcOH (25 mL) was added Fe powder (400 mg), and the reaction stirred at 70° C. for 2 h. The reaction was filtered hot through a pad of celite, washed with DCM (50 mL) and the volatiles removed under reduced pressure. Purification by column chromatography (SiO₂, eluent gradient: 0-100% EtOAC in iso-hexane) afforded the title compound (470 mg, 57%) as a solid.

¹H NMR (400.1 MHz, DMSO-d₆) δ_{H} 0.51 (m, 4H), 0.98 (m, 1H), 1.55 (m, 2H), 1.92 (m, 6H), 3.66 (d, 1H), 4.17 (quintet, 1H), 7.64 (s, 1H), 10.79 (s, 1H); MS m/z 293 [M+H]⁺.

### Intermediate 16

### Methyl(2R)-[(2-chloro-5-nitropyrimidin-4-yl)(cyclopentyl)amino](cyclopropyl)acetate

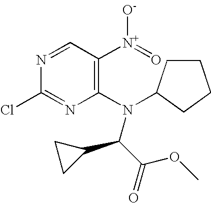

To a solution of methyl(2R)-(cyclopentylamino)(cyclopropyl)acetate (Intermediate 17; 1.65 g, 8.36 mmol) in acetone (40 mL) was added K₂CO₃ (1.18 g, 8.53 mmol) and 2,4-dichloro-5-nitro pyrimidine (1.78 g, 9.20 mmol) and the reaction heated at reflux for 3 h. The reaction was cooled to ambient temperature and the volatiles removed under reduced pressure. The crude reaction mixture was dissolved in EtOAc (40 mL), washed with water (40 mL) and dried (Na₂SO₄) and the volatiles removed under reduced pressure. Purification by column chromatography (SiO₂, eluent gradient: 5-10% EtOAc in iso-hexane) afforded the title compound (1 g, 34%) as a solid.

¹H NMR (400 MHz, DMSO-d₆) δ_{H} 0.28 (m, 1H), 0.46 (m, 1H), 0.72 (m, 2H), 1.49 (m, 2H), 1.68 (m, 3H), 1.77 (m, 3H), 2.01 (m, 1H), 3.60 (m, 2H), 3.69 (s, 3H), 8.85 (s, 1H); MS m/z 355 [M+H]⁺.

### Intermediate 17

### Methyl(2R)-(cyclopentylamino)(cyclopropyl)acetate

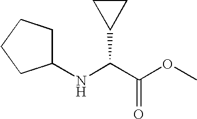

To a solution of methyl(2R)-amino(cyclopropyl)acetate (2.80 g, 19.6 mmol) in DCM (25 mL) was added cyclopentanone (1.53 mL, 17.2 mmol) and the reaction stirred at ambient temperature for 15 minutes. NaOAc (1.4 g, 17.2 mmol) and NaB(OAc)₃H (5.4 g, 25.4 mmol) were then added and the reaction stirred at ambient temperature for a further 20 h. The reaction was diluted with DCM (50 mL), washed with NaHCO₃ (50 mL, saturated aqueous) then dried (MgSO₄). After filtration the volatiles were removed under reduced pressure to afford the title compound (3.1 g, 91%) as an oil.

¹H NMR (400 MHz, DMSO-d₆) δ_{H} 0.01 (m, 2H), 0.18 (m, 2H), 0.67 (m, 1H), 0.98 (m, 2H), 1.18 (m, 2H), 1.36 (m, 4H), 2.44 (d, 1H), 2.69 (quintet, 1H), 3.40 (s, 3H).

### Intermediate 18

### 4-{[(7R)- 8-Cyclopentyl-5-methyl-7-propyl-5,6,7,8-tetrahydropteridin-2-yl]amino-3-methoxybenzoic acid

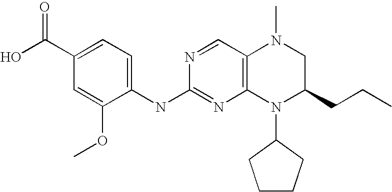

Intermediate 18 was prepared in a manner analogous to Intermediate 11, starting with methyl D-norvalinate as the amino ester.

¹H NMR (400 MHz, DMSO-d₆) δ_{H} 0.92 (t, 3H), 1.42 (m, 4H), 1.67 (m, 6H), 1.87 (m, 3H), 2.00 (m, 2H), 2.78 (s, 3H), 3.70 (d, 1H), 3.94 (s, 3H), 4.67 (m, 1H), 7.30 (s, 1H), 7.51 (s, 1H), 7.56 (d, 1H), 8.31 (s, 1H); MS m/z 426.5 [M+H]⁺.

### Intermediate 19

### 4-[[(7R)-8-Cyclopentyl-7-ethyl-5-methyl-6-oxo-7H-pteridin-2-yl]amino]-3-ethoxy-N-(1-methyl-4-piperidyl)benzamide

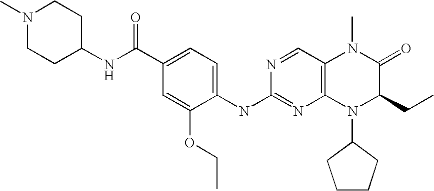

(7R)-2-chloro-8-cyclopentyl-7-ethyl-5-methyl-7H-pteridin-6-one (WO2004076454, 250 mg, 0.85 mmol), 3-ethoxy-N-(1-methyl-4-piperidyl)-4-amino-benzamide (Intermediate 20; 236 mg, 0.85 mmol), and p-toluenesulfonic acid (405 mg, 2.13 mmol) were dissolved in (2R/S)-4-methyl-2-pentanol (4 mL) and stirred at reflux for 24 h. The reaction mixture was then cooled, and poured onto SCX-2 column. The SCX-2 column was washed with MeOH (20 mL) and the crude product was eluted from the SCX-2 column with NH₃ (20 mL, 7M in MeOH). Purification by column chromatography (SiO₂, gradient eluent: 0-10% NH₃ [7M in MeOH] in DCM) afforded the title compound (109 mg, 24%) as a solid.

¹H NMR (400 MHz, CDCl₃) δ_{H} 0.88 (t, 3H), 1.50 (t, 3H), 1.60-1.76 (m, 6H), 1.81-1.91 (m, 4H), 1.98-2.19 (m, 6H), 2.30 (s, 3H), 2.82 (d, 2H), 3.33 (s, 3H), 3.95-4.02 (m, 1H), 4.21 (q, 3H), 4.40-4.45 (m, 1H), 5.89 (d, 1H), 7.21-7.24 (m, 1H), 7.40 (d, 1H), 7.61 (s, 1H), 7.68 (s, 1H), 8.54 (d, 1H); MS m/z 536 [M+H]⁺.

### Intermediate 20

### 3-Ethoxy-N-(1-methyl-4-piperidyl)-4-amino-benzamide

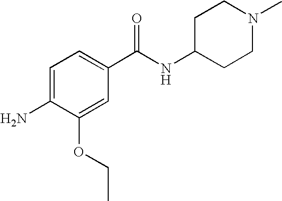

3-Ethoxy-N-(1-methyl-4-piperidyl)-4-nitro-benzamide (Intermediate 21; 270 mg, 0.88 mmol) was dissolved in MeOH (4 mL). Pd (27 mg, 10% on carbon) was added and the flask subjected to H₂ gas at 4 bar pressure with vigorous stirring at ambient temperature for 16 h. The reaction mixture was then filtered through celite washing with MeOH (50 mL). The crude material was absorbed onto an SCX-2 column and washed with MeOH (30 mL). The product was then eluted from the SCX-2 column with NH₃ (30 mL, 7M in MeOH) and the volatiles removed under reduced pressure to afford the title compound (249 mg, 90%) as an oil.

MS m/z 278 [M+H]⁺.

### Intermediate 21

### 3-Ethoxy-N-(1-methyl-4-piperidyl)-4-nitro-benzamide

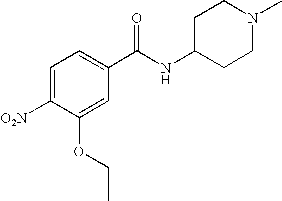

3-Ethoxy-4-nitro-benzoic acid (WO2001077101; 280 mg, 1.33 mmol), 1-methyl-4-aminopiperidine (152 mg, 1.33 mmol) and DIPEA (499 uL, 2.79 mmol) were dissolved in DCM (3 mL) and DMF (1 mL). HATU (532 mg, 1.40 mmol) was added and the resulting solution was stirred at ambient temperature for 5 h. 2N NaOH (20 mL) was added and the aqueous phase extracted with DCM (2×30 mL). The combined organic phases were washed with water (100 mL), brine (100 mL), dried (MgSO₄) and the solvent removed under reduced pressure. Purification by column chromatography (SiO₂, gradient eluent: 1-5% NH₃ [7M in MeOH] in DCM) afforded the title compound (287 mg, 71%) as a solid.

¹H NMR (400 MHz, DMSO-d₆): δ_{H} 1.37 (t, 3H), 1.59 (m, 2H), 1.79 (m, 2H), 1.96 (m, 2H), 2.18 (s, 3H), 2.79 (m, 2H), 3.74 (m, 1H), 4.29 (q, 2H), 7.55 (dd, 1H), 7.68 (d, 1H), 7.93 (d, 1H), 8.47 (d, 1H); MS m/z 308 [M+H]⁺.

### Biological Assays for Inhibition of PLK

The following assay was used to measure the effects of the compounds of the present invention as Plk kinase inhibitors.

### In Vitro Plk1 Enzyme Assay

The assay uses Scintillation Proximity Assay (SPA) technology (Antonsson et al., Analytical Biochemistry, 1999, 267: 294-299) to determine the ability of test compounds to inhibit phosphorylation by recombinant Plk1. The full-length Plk1 protein is expressed in insect cells as an N-terminal 6His tag fusion and purified by standard Nickel chelate purification techniques using the His tag.

The amino terminal fragment of Cdc25C (encoding residues 1-165) is expressed in E. coli as a GST fusion and purified using the GST tag by standard purification techniques.

Test compounds were prepared as 10 mM stock solutions in dimethyl sulphoxide (DMSO) and diluted into water as required to give a range of final assay concentrations. Aliquots (5 μl) of each compound dilution were dispensed into a well of a 384-well flat bottom white polystyrene plate (Matrix, Catalogue No. 4316). A 35 μl mixture of recombinant purified Plk1 enzyme (12 ng/well), purified GST-Cdc25C (150 ng/well), adenosine triphosphate (ATP; 64 nM), ³³P-labelled adenosine triphosphate (³³P-ATP; 60 nCi/well) in a buffer solution [comprising 50 mM HEPES pH7.5 buffer, 10 mM manganese chloride (MnCl₂), 1 mM dithiothreitol (DTT), 1 mg/ml bovine serum albumin (BSA), 100 μM sodium vanadate (Na₃VO₄), 100 μM sodium fluoride (NaF) and 10 mM sodium glycerophosphate] was added and the reactions incubated at ambient temperature for 90 minutes.

Reactions were stopped by addition of EDTA (110 mM) and the Cdc25C substrate captured via its GST tag to anti-GST antibody (Molecular Probes, Cat No A-5800) coated Protein A PVT SPA beads (Amersham Biosciences, Catalogue No. RPQ0019; 250 μg/well) in 50 mM HEPES pH7.5 buffer containing 0.05% (w/v) sodium azide and incubated for up to 2 hours, followed by the addition of 20 μl of 4M caesium chloride (final assay concentration of 1M). Plates were then left in the dark overnight before counting on a Packard TopCount NXT.

Radiolabelled phosphorylated substrate is formed in situ as a result of Plk1 mediated phosphorylation. The SPA beads contain a scintillant that can be stimulated to emit light. This stimulation only occurs when a radiolabelled phosphorylated substrate is bound to the surface of the coated SPA bead causing the emission of blue light that can be measured on a scintillation counter. Accordingly, the extent of Plk1 mediated Cdc25C phosphorylation was assessed. The raw assay data were then analysed by non-linear regression analysis and Plk1 enzyme inhibition for a given test compound is expressed as an IC50 value.

### Cellular Assay

Chromosome condensation in mitosis is accompanied by phosphorylation of histone H3 on serine 10. Dephosphorylation begins in anaphase and ends at early telophase, thus histone H3 serine 10 phosphorylation acts as an excellent mitotic marker and is used to determine the ability of compounds of the present invention to block cells in mitosis.

Cells of the human colon tumour cell line HT29 were seeded into 96 well black plates (Costar, Catalogue No 3904) in phenol red free Dulbecco's Modified Eagles Medium (DMEM) supplemented with 10% (v/v) FCS and 1% (v/v) L-Glutamine and incubated overnight at 37° C. Test compounds were solubilised in DMSO, diluted to give a range of final assay concentrations, added to cells and incubated for 24 h at 37° C. After 24 hours, cells were fixed in 3.7% (v/v) formaldehyde then permeabilised and blocked for 10 minutes in 100 μl 0.5% (v/v) Triton X-100, 1% (w/v) bovine serum albumin (BSA) in phosphate buffered saline (PBS). After washing with PBS, 50 μl primary antibody (1:500 dilution of rabbit anti-phosphohistone H3 (Upstate Catalogue No 06-570) in 1% BSA, 0.05% Tween 20) was added to the cells that were left for 1 hour at room temperature. Cells were again washed with PBS and incubated with 50 μl secondary antibody (1:1000 Alexa Fluor 488 goat anti-rabbit (Molecular Probes Cat No A-11008) and Hoechst (1:10000) diluted in PBS 0.05% (v/v) Tween 20 and left for 1 hour at room temperature in the dark. Cells were washed with PBS then covered with fresh PBS and stored at 4° C. until analysis. Images are acquired and analysed in an automated manner using the Cellomics ArrayScan II or VTi. In this assay both hoechst and phosphohistone H3 staining are measured. Hoechst staining generates a valid cell count while phosphohistone H3 staining determines the number of mitotic cells. Inhibition of Plk leads to an increase in the population of histone H3 Ser10 positive cells, indicating inhibition of proliferation is brought about primarily by arrest of cells in the mitotic phase of the cell cycle. The raw assay data were analysed by non-linear regression analysis and used to determine an IC50 value for each compound.

Inhibition data for example compounds of this invention are summarised in Table 1 where potency is reported in the following context

A indicates IC50 value in the range less than 3 μM

B indicates IC50 value in the range greater than 3 and less than 6 μM

C indicates IC50 value in the range greater than 6 and less than 15 μM

For example, Example 6 was measured to have IC50 value of 256 nM in the Cell assay, and an IC50 value of 699 nM in the enzyme assay.

TABLE 1
Activity of Plk inhibitors

| Example No | Kinase Inhibition |
|---|---|
| 1 | A |
| 2 | A |
| 3 | C |
| 4 | B |
| 5 | A |
| 6 | A |
| 7 | A |
| 8 | A |

1. A compound of formula (I): 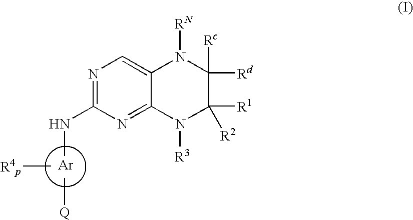
wherein
R¹, R² each independently represents hydrogen, an optionally substituted C₁₋₆alkyl group or an optionally substituted C₃₋₆cycloalkyl group, or R¹ and R² together with the carbon atom to which they are attached form a 3- to 6-membered saturated or unsaturated ring optionally comprising 1 to 2 heteroatoms;
R³ represents hydrogen, an optionally substituted C₁₋₁₂alkyl group, an optionally substituted C₂₋₁₂alkenyl group, an optionally substituted C₂₋₁₂alkynyl group, an optionally substituted C₆₋₁₄aryl group, an optionally substituted C₃₋₁₂cycloalkyl group, an optionally substituted C₃₋₁₂cycloalkenyl group, an optionally substituted C₇₋₁₂polycycloalkyl group, an optionally substituted C₇₋₁₂polycycloalkenyl group, an optionally substituted C₅₋₁₂spirocycloalkyl group, an optionally substituted C₃₋₁₂heterocycloalkyl group comprising 1 or 2 heteroatoms, or an optionally substituted C₃₋₁₂heterocycloalkenyl group comprising 1 or 2 heteroatoms;
R^{c}, R^{d} each independently represents hydrogen, an optionally substituted C₁₋₆alkyl group or an optionally substituted C₃₋₆cycloalkyl group, or R^{c} and R^{d} together with the carbon atom to which they are attached form a 3- to 6-membered saturated or unsaturated ring optionally comprising 1 to 2 heteroatoms; or
optionally one of R¹ and R³, or R² and R³, or R¹ and R^{c}, or R² and R^{d} together represent a saturated or unsaturated C₁₋₄alkyl bridge optionally comprising 1 heteroatom;
R⁴ each independently represent —CN, hydroxy, —NR⁶R⁷, halogen, an optionally substituted C₁₋₆alkyl group, an optionally substituted C₃₋₆cycloalkyl group, an optionally substituted C₂₋₆alkenyl group, an optionally substituted C₂₋₆alkynyl group, an optionally substituted C₁₋₅alkyloxy group, an optionally substituted C₃₋₆cycloalkyloxy group, an optionally substituted C₂₋₅alkenyloxy group, an optionally substituted C₂₋₅alkynyloxy group, an optionally substituted C₁₋₆alkythio group, an optionally substituted C₁₋₆alkylsulphoxo group or an optionally substituted C₁₋₆alkylsulphonyl group;
p is 0, 1 or 2;
Q¹ is —C(═X)—NR^{a}R^{b}, —NR^{a2}R^{b2}, —S(O)₂—NR^{a3}R^{a3}, S(O)ₖ—R^{a4}, —C(═X)—OR^{a5}, —OR^{a6};
k is 0, 1 or 2;
R^{a} represents H or an optionally substituted C₁₋₆alkyl group, and R^{b} represents -Lₙ-R⁵ₘ, or R^{a} and R^{b} together with the nitrogen atom to which they are attached form a 3- to 7-membered saturated or unsaturated heterocyclic ring optionally comprising 1 to 2 additional heteroatoms;
R^{a2} represents H or an optionally substituted C₁₋₆alkyl group, and R^{b2} represents -Lₙ-R⁵ₘ, or R^{a2} and R^{b2} together with the nitrogen atom to which they are attached form a 3- to 7-membered saturated or unsaturated heterocyclic ring optionally comprising 1 to 2 additional heteroatoms;
R^{a3} represents H or an optionally substituted C₁₋₆alkyl group, and R^{b3} represents -Lₙ-R⁵ₘ, or R^{a3} and R^{b3} together with the nitrogen atom to which they are attached form a 3- to 7-membered saturated or unsaturated heterocyclic ring optionally comprising 1 to 2 additional heteroatoms;
R^{a4} represents -Lₙ-R⁵ₘ;
R^{a5} represents -Lₙ-R⁵ₘ;
R^{a6} represents -Lₙ-R⁵ₘ;
L represents a linker selected from optionally substituted C₂₋₁₀alkyl, optionally substituted C₂₋₁₀alkenyl, optionally substituted C₆₋₁₄aryl, optionally substituted —C₂₋₄alkyl-C₆₋₁₄aryl, optionally substituted —C₆₋₁₄aryl-C₁₋₄alkyl, optionally substituted C₃₋₁₂cycloalkyl and optionally substituted heteroaryl comprising 1 or 2 nitrogen atoms;
n is 0 or 1
m is 1 or 2
R⁵ represents a group selected from among optionally substituted morpholinyl, piperidinyl, piperazinyl, piperazinylcarbonyl, pyrrolidinyl, tropenyl, diketomethylpiperazinyl, sulphoxomorpholinyl, sulphonylmorpholinyl, thiomorpholinyl, azacycloheptyl and —NR⁸R⁹;
R⁶, R⁷ each independently represents hydrogen or an optionally substituted C₁₋₄alkyl group;
R⁸, R⁹ each independently represents hydrogen, C₁₋₆alkyl, —C₁₋₄alkyl-C₃₋₁₀cycloalkyl, C₃₋₁₀cycloalkyl, C₆₋₁₄aryl, —C₁₋₄alkyl-C₆₋₁₄aryl, pyranyl, pyridinyl, pyrimidinyl, C₁₋₄alkyloxycarbonyl, C₆₋₁₄arylcarbonyl, C₁₋₄alkylcarbonyl, C₆₋₁₄arylmethyloxycarbonyl, C₆₋₁₄arylsulphonyl, C₁₋₄alkylsulphonyl or C₆₋₁₄aryl-C₁₋₄alkylsulphonyl;
X is O, S or H₂;
Ar represents a 5- or 6-membered aromatic or heteroaromatic ring optionally comprising at least one ring heteroatom selected from nitrogen, oxygen and sulphur; and
R^{N} represents hydrogen, —NH₂, —OH, —CN, —C≡CH, —C(═O)NH₂, C₁₋₃alkyl, C-₁₋₃alkylamino, C₁₋₃alkylthio or C₁₋₃alkyloxy,
or optionally the pharmacologically acceptable acid addition salts thereof.

2. A compound according to claim 1 wherein R^{N} represents C₁₋₃alkyl.

3. A compound according to claim 1 wherein R^{N} represents methyl or ethyl.

4. A compound according to claim 1 wherein Q is —C(═X)—NR^{a}R^{b} and X is O or CH₂.

5. A compound of formula (II): 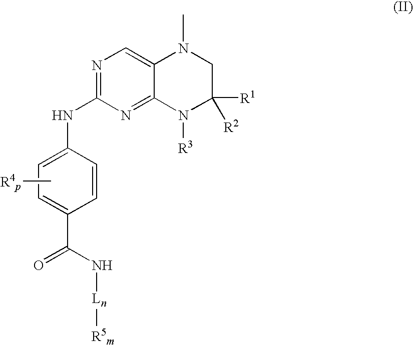
wherein
R¹, R² each independently represents hydrogen or an optionally substituted C₁₋₆alkyl group, or
R¹ and R² together with the carbon atom to which they are attached form a 3- to 6-membered saturated or unsaturated ring optionally comprising 1 to 2 heteroatoms;
R³ represents hydrogen, an optionally substituted C₁₋₁₂alkyl group, an optionally substituted C₂₋₁₂alkenyl group, an optionally substituted C₂₋₁₂alkynyl group, an optionally substituted C₆₋₁₄aryl group, an optionally substituted C₃₋₁₂cycloalkyl group, an optionally substituted C₃₋₁₂cycloalkenyl group, an optionally substituted C₇₋₁₂polycycloalkyl group, an optionally substituted C₇₋₁₂polycycloalkenyl group, an optionally substituted C₅₋₁₂spirocycloalkyl group, an optionally substituted C₃₋₁₂heterocycloalkyl group comprising 1 or 2 heteroatoms, or an optionally substituted C₃₋₁₂heterocycloalkenyl group comprising 1 or 2 heteroatoms, or
R¹ and R³ or R² and R³ together represent a saturated or unsaturated C₃₋₄alkyl bridge optionally comprising 1 heteroatom;
R⁴ each independently represent —CN, hydroxy, —NR⁶R⁷, halogen, an optionally substituted C₁₋₆alkyl group, an optionally substituted C₃₋₆cycloalkyl group, an optionally substituted C₂₋₆alkenyl group, an optionally substituted C₂₋₆alkynyl group, an optionally substituted C₁₋₅alkyloxy group, an optionally substituted C₃₋₆cycloalkyloxy group, an optionally substituted C₂₋₅alkenyloxy group, an optionally substituted C₂₋₅alkynyloxy group, an optionally substituted C₁₋₆alkythio group, an optionally substituted C₁₋₆alkylsulphoxo group or an optionally substituted C₁₋₆alkylsulphonyl group;
p is 0, 1 or 2;
L represents a linker selected from optionally substituted C₂₋₁₀alkyl, optionally substituted C₂₋₁₀alkenyl, optionally substituted C₆₋₁₄aryl, optionally substituted —C₂₋₄alkyl-C₆₋₁₄aryl, optionally substituted —C₆₋₁₄aryl-C₁₋₄alkyl, optionally substituted C₃₋₁₂cycloalkyl and optionally substituted heteroaryl comprising 1 or 2 nitrogen atoms;
n is 0 or 1
m is 1 or 2
R⁵ represents a group selected from among optionally substituted morpholinyl, piperidinyl, piperazinyl, piperazinylcarbonyl, pyrrolidinyl, tropenyl, diketomethylpiperazinyl, sulphoxomorpholinyl, sulphonylmorpholinyl, thiomorpholinyl, azacycloheptyl and —NR⁸R⁹;
R⁶, R⁷ each independently represents hydrogen or an optionally substituted C₁₋₄alkyl group; and
R⁸, R⁹ each independently represents hydrogen, C₁₋₆alkyl, —C₁₋₄alkyl-C₃₋₁₀cycloalkyl, C₃₋₁₀cycloalkyl, C₆₋₁₄aryl, —C₁₋₄alkyl-C₆₋₁₄aryl, pyranyl, pyridinyl, pyrimidinyl, C₁₋₄alkyloxycarbonyl, C₆₋₁₄arylcarbonyl, C₁₋₄alkylcarbonyl, C₆₋₁₄arylmethyloxycarbonyl, C₆₋₁₄arylsulphonyl, C₁₋₄alkylsulphonyl and C₆₋₁₄aryl-C₁₋₄alkylsulphonyl, or optionally the pharmacologically acceptable acid addition salts thereof.

6. A compound according to claim 1 wherein R¹ and R² may be identical or different and represent hydrogen or a C₁-C₆alkyl group optionally substituted by at least one substituent selected from C₁₋₃alkyloxy, C₁₋₃alkylthio, C₁₋₃alkyl-S(O)₂, C₁₋₃alkylamino and di-(C₁₋₃alkyl)amino.

7. A compound according to claim 6 wherein R¹ and R² are different and wherein one of R¹ or R² represents hydrogen and the other represents a methyl or ethyl group.

8. A compound according to claim 1 wherein R³ represents isopropyl, isobutyl, isopentyl, cyclopentyl, phenyl or cyclohexyl.

9. A compound according to claim 1 wherein when p is 1, R⁴ represents methoxy, methyl, ethoxy, ethyl, propargyloxy, chlorine.

10. A compound according to claim 1 wherein when p is 2, each R⁴ may be the same or different and selected from methoxy, methyl, ethoxy, ethyl, propargyloxy, chlorine or fluorine.

11. A compound according to claim 1 wherein, when p is 2 and when each R⁴ is adjacent, both R⁴ together with the aromatic ring atoms to which they are attached form a 4- to 7-member unsaturated ring optionally comprising 1 to 2 heteroatoms.

12. A compound according to claim 1 wherein when n is 1, L represents an optionally substituted a C₂₋₁₀alkyl linker.

13. A compound according to claim 12 wherein when n is 1, L represents —C(CH₃)₂—CH₂— or —CH₂—C(CH₃)₂—CH₂—.

14. A compound according to claim 1 wherein m is 1 and R⁵ represents NR⁸R⁹ or a piperidinyl, morpholinyl, pyrrolidinyl, sulphoxomorpholiny, piperazinyl, thiomorpholinyl or tropenyl each optionally substituted by one or more groups as defined for R⁸.

15. A compound according to claim 14 wherein R⁸ represents methyl, ethyl or propyl, and R⁹ represents methyl, ethyl or propyl.

16. A process for preparing a compound of general formula (II), 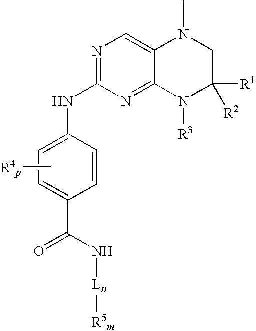
wherein R¹-R⁵, m, n and L are as defined in claim 5, comprising reacting a compound of general formula (III) 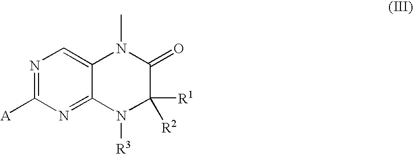
wherein R¹-R³ are as hereinbefore defined and A is a leaving group, with an optionally substituted compound of general formula (IV): 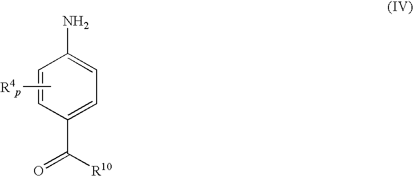
wherein R⁴ is as hereinbefore defined; and R¹⁰ denotes OH, NH-Lₘ-R⁵ₙ, OMe, OEt, to give a product of general formula (V) 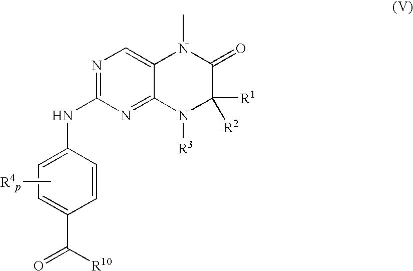
wherein R¹ to R⁴ is as hereinbefore defined; and R¹⁰ denotes OH, NH-Lₘ-R⁵ₙ, OMe or OEt, and
a) when R¹⁰ denotes NH-Lₘ-R⁵ₙ, reducing the compound of formula (V) to give a compound of formula (II), or
b) when R¹⁰ denotes OH, OMe or OEt either
i) optionally after previous hydrolysis of the ester group —COR¹⁰, reacting the compound of formula (V) with an amine of general formula (VI):
NH₂-Lₘ-R⁵ₙ (VI)
wherein R⁵ is as hereinbefore defined,
to give a compound of formula (Va) 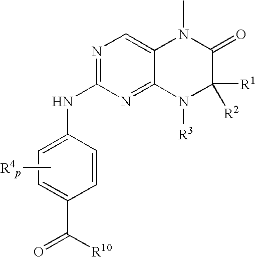
wherein R¹ to R⁴ is as hereinbefore defined; and R¹⁰ denotes NH-Lₘ-R⁵ₙ,
and reducing the compound of formula (Va) to give a compound of formula (II), or
ii) optionally after previous hydrolysis of the ester group —COR¹⁰, reducing the compound of formula (V) to give a compound of formula (VII) 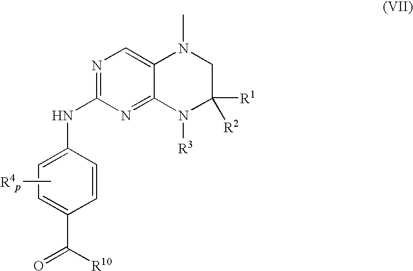
wherein R¹ to R⁴ is as hereinbefore defined; and R¹⁰ denotes OH, OMe or OEt,
and reacting the compound of formula (VII), optionally after previous hydrolysis of the ester group —COR¹⁰, with an amine of general formula (VI):
NH₂-Lₘ-R⁵ₙ (VI)
wherein R⁵ is as hereinbefore defined,
to give a compound of formula (II).

17. A pharmaceutical composition comprising a compound of formula (I) as defined in claim 1, or a pharmaceutically acceptable salt thereof, in association with a pharmaceutically acceptable adjuvant, diluent or carrier.

18. A process for the preparation of a pharmaceutical composition as claimed in claim 17 which comprises mixing a compound of formula (I), or a pharmaceutically acceptable salt thereof, with a pharmaceutically acceptable adjuvant, diluent or carrier.

19. (canceled)

20. (canceled)

21. (canceled)

22. A method of treating cancer which comprises administering to a patient in need thereof a therapeutically effective amount of a compound of formula (I), or a pharmaceutically acceptable salt thereof, as claimed in claim 1.

23. A method of modulating polo-like kinase (Plk) activity which comprises administering to a patient in need thereof a therapeutically effective amount of a compound of formula (I), or a pharmaceutically acceptable salt thereof, as claimed in claim 1.

24. (canceled)
